(19) United States
(12) Patent Application Publication
(10) Pub. No.: US 2024/0398793 A1
(43) Pub. Date: Dec. 5, 2024

(54) Methods of Treating Lymphedema

(71) Applicants: Celltaxis, LLC, Atlanta, GA (US); The Board of Trustees of the Leland Stanford Junior University, Stanford, CA (US)

(72) Inventors: Stanley Glenn ROCKSON, Palo Alto, CA (US); Mark R. NICOLLS, Palo Alto, CA (US); Xinguo JIANG, Palo Alto, CA (US); Wen TIAN, Redwood City, CA (US); Eric B. SPRINGMAN, Atlanta, GA (US); Eileen HEFFERNAN, Osterville, MA (US)

(21) Appl. No.: 18/736,660

(22) Filed: Jun. 7, 2024

Related U.S. Application Data
(63) Continuation of application No. PCT/US2022/052230, filed on Dec. 8, 2022.
(60) Provisional application No. 63/287,748, filed on Dec. 9, 2021.

(51) Int. Cl. A61K 31/4995; A61K 31/192; A61K 45/06

(52) U.S. Cl. CPC A61K 31/4995; A61K 31/192; A61K 45/06

(57) ABSTRACT

Described are methods of treating lymphedema, methods of reducing dermal thickening, methods of improving skin turgor, structure, histology and/or function, and/or methods of improving lymphatic flow and/or vascular function in a patient in need thereof, comprising administering to said patient an effective amount of a selective LTA4H inhibitor.

### RELATED APPLICATIONS

This application is a continuation of International Application No. PCT/US22/52230, which designated the United States and was filed on Dec. 8, 2022, published in English, which claims the benefit of U.S. Provisional Application No. 63/287,748 filed Dec. 9, 2021. The entire contents of the above-referenced applications are incorporated by reference herein.

### BACKGROUND OF THE INVENTION

Lymphedema is a debilitating chronic condition with no definitive pharmacologic treatment. Although mortality is low, lymphedema-related hospitalizations are a significant burden to the U.S. healthcare system⁴. Lymphedema affects as many as 200 million patients worldwide and at least 3 million in the U.S.⁶. However, because lymphedema is underrecognized and under-documented, it is highly likely that the currently accepted rates of incidence and prevalence underestimate its magnitude⁷.

Lymphedema occurs when impaired fluid transport through the lymphatic circulation leads to non-resolving, progressively nonpitting fluid retention^{9, 10}. The disease is caused by relative lymphatic vascular insufficiency¹¹. The natural history of lymphedema is defined by increasing limb girth, fibrosis, inflammation, abnormal fat deposition and marked cutaneous pathology that increases the risk of recurrent skin infections¹². Lymphedema can substantially affect the daily quality of life of patients, as, in addition to aesthetic concerns, it can cause discomfort and affects the ability to carry out daily tasks.

Lymphedema is characterized by an imbalance of growth factors¹³ and by the presence of persistent tissue inflammation¹⁰. While therapeutic approaches that foster reparative lymphangiogenesis might, in the future, have the potential to reverse the pathology of lymphedema, the relationship between persistent inflammation and impaired lymphangiogenesis has not, until recently, been sufficiently explored¹⁴.

In a prospective search for informed pharmacological approaches, the mechanistic role of leukotriene B4 (LTB4) in the molecular pathogenesis of both human and murine experimental lymphedema has been identified^{14, 15}. Prior translational investigation of a murine model of human lymphedema¹⁶ suggested a role for leukotrienes, both in the pathogenesis of lymphedema and as a potential therapeutic target¹⁶. Ketoprofen¹⁷, an NSAID agent with a recognized dual anti-inflammatory mechanism of action that includes inhibition of the 5-lipoxygenase (5-LO) pathway¹⁸ has been investigated as therapy for lymphedema. See, for example, U.S. Pat. No. 8,965,708. When ketoprofen was systemically administered to mice with experimental, acquired lymphedema, disease burden was reversed, including remarkable normalization of the pre-treatment lymphedematous histopathology¹⁷. Results of the first human pilot clinical trial of pharmacotherapy for human lymphedema have also been published. This exploratory open-label and subsequent placebo-randomized investigation of an aggregate⁵⁵ adult lymphedema subjects confirmed the ability of the drug to improve skin thickness, measured clinically, and to reverse cutaneous histopathological changes of lymphedema¹⁹. While these results highlight the promise of such approaches to help restore a failing lymphatic circulation, the clinical application is hampered by the concerns over cardiovascular morbidity associated with long-term NSAID use.

U.S. Pat. No. 10,500,178 describes the use of certain inhibitors of LTB4 for the prevention and treatment of lymphedema. In addition, the LTA₄ hydrolase inhibitor, Ubenimex (also referred to in the literature as “bestatin”) has been investigated in a Phase 2 clinical trial for the treatment of lymphedema of the lower limb. The clinical study did not identify an improvement in skin thickness and limb volume and bioimpedance for Ubenimex over placebo.

Currently, there are no approved therapies for the treatment of lymphedema. Thus, there remains a significant need for treatment that can safely and effectively treating this chronic, debilitating condition.

### SUMMARY OF THE INVENTION

The present invention is directed to methods of treating lymphedema, methods of reducing dermal thickening, methods of improving skin turgor, structure, histology and/or function, and/or methods of improving lymphatic flow and/or vascular function in a patient in need thereof, comprising administering to said patient an effective amount of acebilustat or another selective LTA4H inhibitor. The lymphedema that can be treated according to the methods of the invention include, for example, primary and secondary (or acquired) lymphedema. The treatment described herein can result in a reduction in dermal thickening, and/or an improvement in skin turgor, structure, histology or function, and/or increased lymphatic flow and/or vascular function.

The invention encompasses a method of treating lymphedema, such as secondary lymphedema, in a patient in need thereof, comprising administering to said patient an effective amount of acebilustat, wherein the treatment results in a reduction in dermal thickening. In additional aspects, the invention is a method treating upper limb extremity lymphedema in a patient in need thereof comprising administering to said patient an effective amount of acebilustat, wherein the treatment results in a reduction in dermal thickening.

The invention also encompasses a method of treating lymphedema, such as secondary lymphedema, in a patient in need thereof, comprising administering to said patient an effective amount of acebilustat, wherein the treatment results in a reduction in dermal thickening, and/or an improvement in skin turgor, structure, histology and/or function. The invention also encompasses a method of treating upper limb extremity lymphedema in a patient in need thereof comprising administering to said patient an effective amount of acebilustat, wherein the treatment results in an improvement in skin turgor, structure, histology and/or function.

The invention additionally includes methods of treating lymphedema, such as secondary lymphedema, in a patient in need thereof, comprising administering to said patient an effective amount of acebilustat, wherein the treatment results in an improvement in lymphatic flow and/or vascular function. In additional aspects, the invention is a method treating upper limb extremity lymphedema in a patient in need thereof comprising administering to said patient an effective amount of acebilustat, wherein the treatment results in an improvement in lymphatic flow.

The invention also includes a method of treating lymphedema in a patient in need thereof comprising administering to said patient an effective amount of a selective LTA4H inhibitor, wherein the treatment results in a reduction in dermal thickening, wherein the selective LTA4H inhibitor is selective for epoxide hydrolase versus aminopeptidase. In certain aspects, the invention is directed to a method treating upper limb extremity lymphedema in a patient in need thereof comprising administering to said patient an effective amount of a selective LTA4H inhibitor, wherein the treatment results in a reduction in dermal thickening, wherein the selective LTA4H inhibitor is selective for epoxide hydrolase versus aminopeptidase.

The invention also encompasses a method of reducing dermal thickening in a patient in need thereof, the method comprising administering to said patient an effective amount of a selective LTA4H inhibitor, wherein the selective LTA4H inhibitor is selective for epoxide hydrolase versus aminopeptidase. In yet additional embodiments, the invention is directed to a method of reducing dermal thickening in a patient in need thereof, the method comprising administering to said patient an effective amount of acebilustat. The patient can, for example, be a patient suffering from lymphedema, a patient in need of improved skin softness, or a patient suffering from scleroderma.

The invention also includes methods of treating lymphedema, such as secondary lymphedema, in a patient in need thereof, comprising administering to said patient an effective amount of a selective LTA4H inhibitor, wherein the treatment results in an increase in an improvement in skin turgor, structure, histology or function. In yet additional aspects, the invention is directed to a method of improving skin turgor, structure, histology or function in a patient in need thereof, the method comprising administering to said patient an effective amount of a selective LTA4H inhibitor. The patient can, for example, be a patient suffering from lymphedema, a patient in need of improved skin softness, or a patient suffering from scleroderma.

The invention also includes a method of treating lymphedema, such as secondary lymphedema, in a patient in need thereof, comprising administering to said patient an effective amount of a selective LTA4H inhibitor, wherein the treatment results in an increase in lymphatic flow and/or an improvement in vascular function. In yet additional aspects, the invention is directed to a method of increasing lymphatic flow and/or an improvement in vascular flow in a patient in need thereof, the method comprising administering to said patient an effective amount of a selective LTA4H inhibitor. The patient can, for example, be a patient suffering from lymphedema, a patient in need of improved skin softness, or a patient suffering from scleroderma.

In further aspects, the invention is directed to a method of treating lymphedema in a patient in need thereof comprising administering to said patient an effective amount of a combination of acebilustat or other selective LTA4H inhibitor and a second active agent, wherein the second active agent is selected from the group consisting of a COX-1 inhibitor, a COX-2 inhibitor, a coumarin, an anti-histamine, montelukast and related leukotriene modifiers, an anti-fibrotic compound, a diuretic, a statin, an mTOR inhibitor, and pirfenidone. In yet another embodiment, the second active agent is selected from the group consisting of a COX-1 inhibitor, a COX-2 inhibitor, a coumarin, an anti-histamine, an anti-fibrotic compound, a diuretic, a statin, an mTOR inhibitor, and pirfenidone. In certain aspects, the lymphedema is upper limb extremity lymphedema.

The reduction in dermal thickening can, for example, be measured by dermal ultrasound, skin caliper, or any other method.

The increase in lymphatic flow and/or vascular function can, for example, be measured by lymphoscintigraphy and/or indocyanine green lymphography.

In certain aspects, the lymphedema is secondary lymphedema including, but not limited, in a patient that has previously undergone surgery and/or radiation therapy for cancer. In some examples, the cancer is a solid tumor. In one example, the patient is suffering from suffering breast cancer treatment associated upper limb lymphedema. In another example, the patient is suffering from head and neck area lymphedema.

In some embodiments, the methods comprise administering a selective LTA4H inhibitor. A selective LTA4H inhibitor is an agent or compound that has more selectivity for inhibiting LTA4H than other aminopeptidase enzymes and/or an agent that has more selectivity for inhibiting the epoxide hydrolase activity of LTA4H than the aminopeptidase activity of LTA4H. In the context of LTA4H inhibitors, selectivity refers to potency of inhibition. For example, an LTA4H inhibitor is more selective for epoxide hydrolase activity than aminopeptidase activity when the IC₅₀ for epoxide hydrolase inhibition is lower than the IC₅₀ for aminopeptidase activity. In one embodiment, the LTA4H inhibitor is at least about 2 times more selective for LTA4H than other aminopeptidase enzymes, a group including, but not limited to, Aminopeptidase N, Aminopeptidase M, Aminopeptidase P, Leucine Aminopeptidase. In another embodiment, the LTA4H inhibitor is at least about 1.5 times more selective for inhibiting epoxide hydrolase activity versus inhibiting aminopeptidase activity of LTA4H. In additional aspects, the LTA4H inhibitor is at least about 1.5 or about 2 times more selective for inhibiting the epoxide hydrolase activity of LTA4H versus inhibiting aminopeptidase activity of LTA4H. In yet further embodiments, the LTA4H inhibitor is at least 2 times more selective for LTA4H than other aminopeptidases and more than about 1.5 times more selective for epoxide hydrolase activity than aminopeptidase activity of LTA4H.

The invention also includes a method of reducing the incidence of soft-tissue infection, e.g., cellulitis, in lymphedema patients, comprising administering acebilustat or other selective LTA4H inhibitor.

In certain aspects, the methods comprise administering to the patient an effective amount of acebilustat. For example, the acebilustat can be administered at least once a day, for example orally. In certain embodiments, the methods comprise orally administering to patients acebilustat at a total daily dose of about 200 mg or less, about 150 mg or less, about 100 mg or less, about 50 mg or less, from about 50 mg to about 100 mg, about 100 mg, or about 50 mg.

In other aspects, the methods comprising topical administration of acebilustat or other selective LTA4H inhibitor.

### DETAILED DESCRIPTION OF THE INVENTION

A description of preferred embodiments of the invention follows.

As used herein, the words “a” and “an” are meant to include one or more unless otherwise specified. For example, the term “an additional therapeutic agent” encompasses both a single additional therapeutic agent and a combination of two or more additional therapeutic agents.

It is to be understood that when the range of the dose or amount of a drug or active ingredient (e.g., acebilustat) is described as “between” a low end of the range and “between” a high end of the range, the range is meant to include both, the low end and the high end as well as doses in between the low and high ends. For example, for “a dose between about 50 mg and about 100 mg,” it is to be understood that the range includes the low end of the range, about 50 mg, and the high end of the range, about 100 mg, as well as the doses in between, for example, about 75 mg. In addition, “a dose of about 50 mg or less” is intended to include the about 50 mg dose as well as doses less than about 50 mg.

The term “about” as used herein, in reference to a numerical value or range, allows for a degree of variability in the value or range, for example, within 10%, within 5%, or within 4%, or within 2% of the value or range, or within 1% of the value or range.

The present invention is directed to the use of acebilustat as well as other selective LTA4H inhibitors to inhibit the enzymatic production of LTB4 in lymphedema patients, for example, patients with upper extremity lymphedema, and, thereby, to reverse the pathological cutaneous stigmata of the disease that are responsible for morbidity, loss of function, and of quality-of-life.

Lymphedema, as used herein, is edema of a region or regions of the body due to lymphatic maldevelopment (primary lymphedema) or to obstruction, disruption or dysfunction (secondary or acquired lymphedema) of lymphatic vessels. Symptoms and signs can comprise varying degrees of brawny, fibrous, non-pitting edema in one or more regions of the body.

Primary lymphedemas are constitutional and relatively less common than the secondary forms. They vary in phenotype and patient age at presentation. The methods of the invention are applicable to these primary forms, although it will be understood by one of skill in the art that treatment may be more efficacious in some forms than others due to the differing disease etiologies. Primary forms of lymphedema include, without limitation Milroy's disease, Meige disease, (lymphedema praccox), lymphedema distichiasis, lymphedema tarda, etc., as well as other genetic syndromes having prominent lymphedema, such as Turner's syndrome and Hennekam syndrome. For example, congenital lymphedema appears at birth or within months thereafter, and may be due to lymphatic aplasia or hypoplasia. Milroy's disease is an autosomal dominant familial form of congenital lymphedema attributed to FLT4 gene mutations and associated with edema and, sometimes, diarrhea and/or hypoproteinemia due to a protein-losing enteropathy caused by intestinal lymphangiectasia. Lymphedema distichiasis is an autosomal dominant familial form of lymphedema praecox attributed to mutations in a transcription factor gene (FOXC2) and associated with extra eyelashes (distichiasis), and edema of legs, arms, and sometimes the face. Lymphedema tarda occurs after age 35. Both familial and sporadic forms exist; the genetic basis of both is unknown. Clinical findings are similar to those of lymphedema praecox but may be less severe. Hereditary lymphedema type II (Meige disease, lymphedema praecox) develops around puberty or shortly thereafter in most individuals. This is the most common type of primary lymphedema. In addition to lymphedema of the legs, other areas of the body such as the arms, face and larynx may be affected. Some individuals may develop yellow nails. Lymphedema is prominent in some other genetic syndromes, including Turner syndrome; yellow nail syndrome, characterized by pleural effusions, chronic lung disease, lymphedema and yellow nails; and Hennekam syndrome, a rare congenital syndrome of generalized lymphatic abnormality, facial anomalies, and intellectual disability. The methods and compositions of the invention can be used to treat any of these primary lymphedemas and their symptoms.

Secondary (acquired) lymphedema is far more common than primary lymphedema. It is commonly caused by surgery (especially lymph node dissection, typically for staging and treatment of cancers), radiation therapy (especially axillary or inguinal), trauma, lymphatic obstruction by a tumor, and, in developing countries, lymphatic filariasis. In certain aspects, the methods described herein are used to treat secondary lymphedema.

In certain embodiments, the methods comprise treatment of patients with established secondary lymphedema, for example, contracted as a result of cancer therapy. It has been estimated that more than 15% of breast cancer survivors experience secondary lymphedema. Surgical removal of lymph nodes or therapeutic radiation of lymph nodes increases the risk of lymphedema. After axillary intervention, 15% to 30% of breast cancer survivors experience clinically relevant lymphedema, but other types of cancer and their associated treatments may cause secondary lymphedema as well. The incidences of lymphedema associated with other malignancies (cancers) were as follows: soft tissue sarcoma 30%, lower extremity melanoma 28%, gynecologic cancer 20%, genitourinary cancer 10%, and head and neck cancers 3%. Lymphedema can also result from increased lymph production in patients with chronic venous insufficiency, congestive heart failure, and other causes of venous hypertension. The methods of the invention are applicable to all such secondary lymphedema patients. In certain aspects, the methods are directed to the treatment of upper limb extremity lymphedema. In an additional aspect, the invention is directed to the treatment of upper limb extremity lymphedema after breast cancer treatment (referred to herein as breast cancer treatment associated upper limb lymphedema). In further aspects, the lymphedema is associated with head and neck cancer.

The cardinal sign of acquired lymphedema is soft-tissue edema, graded in 4 stages. The term “established lymphedema” can refer generically to any of stages 1-3 of the disease, including without limitation the more advanced stages of the disease, e.g., stage 2 and stage 3, where structural changes in affected tissue are observed. In stage 0, the affected region is physically normal, but lymphatic insufficiency can be demonstrated through clinical assessment. In stage 1, the edema is pitting, and the affected area often returns to normal after elevation of the affected limb(s). In stage 2, the edema is pitting, and chronic soft-tissue inflammation causes structural changes in the tissues that accompany the pitting edema. In stage 3, the edema is brawny and irreversible, largely because of chronic soft-tissue structural changes.

Acebilustat and other LTA4H inhibitors have been described, for example, in U.S. Pat. Nos. 7,737,145, 9,820,974, and U.S. Patent Application Publication No. 20100210630A1, the contents of each of which are incorporated by reference herein. The chemical name of acebilustat is 4-{[(1S,4S)-5-({4-[4-oxazol-2-yl-phenoxy]phenyl}methyl)-2,5-diazabicyclo[2.2.1]heptan-2-yl]methyl}benzoic acid (also referred to as CTX-4430). Acebilustat is a potent inhibitor of Leukotriene A4 Hydrolase (LTA4H), the rate-limiting enzyme in production of leukotriene B4 (LTB4).

The methods of the invention comprise administration of an effective dose of acebilustat (also known as CTX-4430) to human patients. In certain aspects, the acebilustat is administered orally. This compound and methods for the preparation thereof have been described in detail in U.S. Pat. Nos. 7,737,145, 9,820,974, and U.S. Patent Application Publication No. 20100210630A1, the contents of each of which are incorporated by reference herein. Acebilustat has the chemical structure shown below:

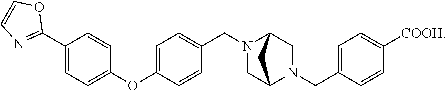 [SMILES: O=C(O)c1ccc(CN2C[C@@H]3C[C@H]2CN3Cc2ccc(Oc3ccc(-c4ncco4)cc3)cc2)cc1]

In vitro, acebilustat inhibits the epoxide hydrolase enzymatic activity of LTA4H with an IC₅₀ of 6.3 ng/mL for LTB4 production. In human whole blood tested ex vivo, acebilustat inhibits LTB4 production with an approximate IC₅₀ of 30.8 ng/mL. Acebilustat 48 ng/mL has also been shown to reduce neutrophil swarming in vitro by 80% in response to factors present in human cystic fibrosis (CF) sputum. In pharmacodynamic studies in humans, acebilustat inhibits LTB4 production with an estimated in vivo EC₅₀ of 93 ng/mL. In CF patients, sputum white blood cells were decreased by 31% from Baseline in all treated subjects (doses of 50 or 100 mg) and by 60% from Baseline in the 100 mg acebilustat group. Sputum neutrophils decreased by 34% in all treated patients (doses of 50 to 100 mg) and by 65% in the 100 mg group. In a recent study Phase II study of adult patients with CF, acebilustat showed promise in reducing the rate of pulmonary exacerbations over the course of 48 weeks of treatment with no evidence of increased risk of infection (described, for example, in U.S. Pat. No. 10,898,484; the contents of which are expressly incorporated by reference herein). This effect was most notable in patients with early disease.

In some examples, an effective amount of acebilustat administered orally can be 200 mg or less. The invention thus encompasses oral administration of about 200 mg or less acebilustat to the patient. In certain aspects, the patient is administered 200 mg of acebilustat; for example, chronic oral administration (e.g., for more than about one day, for at least about one week, for at least about two weeks, for at least about three weeks, for at least about one month, for at least about 2 months, for at least about 3 months, for at least about 4 months, for at least about 5 months, or for at least about 6 months or 24 weeks, and/or throughout the patient's treatment). The invention encompasses oral administration of about 100 mg acebilustat to said patient; for example, chronic oral administration (e.g., for more than about one day, for at least about one week, for at least about two weeks, for at least about three weeks, for at least about one month, for at least about 2 months, for at least about 3 months, for at least about 4 months, for at least about 5 months, or for at least about 6 months or 24 weeks, and/or throughout the patient's treatment). The invention also encompasses administration of 50 mg acebilustat to said patient; for example, chronic oral administration (e.g., for at least about one week, for at least about two weeks, for at least about three weeks, for at least about one month, for at least about 2 months, for at least about 3 months, for at least about 4 months, for at least about 5 months, or for at least about 6 months or 24 weeks, and/or throughout the patient's treatment). Acebilustat can, for example, be administered at a dose of about 50 mg every 12 or 24 hours (or once or twice a day), or at a dose of about 100 mg every 12 or 24 hours (or once or twice a day). In certain aspects, acebilustat is administered at a dose of 100 mg every 24 hours (or once a day). The total daily dose of acebilustat can be a dose that is about 200 mg or less, about 100 mg or less, or about 50 mg or less. The total daily dose of acebilustat can also be from 100 mg to 200 mg, for example about 150 mg. The total daily dose of acebilustat can also be from about 50 mg to about 100 mg, for example, about 75 mg. In certain aspects, the dose of acebilustat is about 25 mg administered once or twice a day or a dose between about 25 and 50 mg administered once or twice a day. Acebilustat can be administered with or without food.

In certain aspects, an effective amount of acebilustat or other selective LTA4H hydrolase inhibitor is administered in an effective amount, wherein the effective amount is less than that which provides maximum inhibition of LTA4H epoxide hydrolase activity. In some examples, the effective amount can provide at least about 50%, at least about 60%, at least about 70%, at least about 80%, or about at least bout 85% inhibition of LTA4H epoxide hydrolase activity, e.g., at least about 50%, at least about 60%, at least about 70%, at least about 80%, or at least about 85% inhibition of LTB4 generation. It has been shown, for example, that a 50 mg dose of acebilustat provides greater than or equal to 63% LTB4 inhibition, that a 100 mg dose of acebilustat provides greater than or equal to about 74% LTB4 inhibition, that a 150 mg dose provides greater than or equal to about 82% LTB4 inhibition and that 200 mg acebilustat provides greater than or equal to about 85% LTB4 inhibition in human pharmacodynamic studies.

In additional aspects, the acebilustat or other selective LTA4 hydrolase inhibitor is administered topically.

The invention also includes methods wherein the acebilustat or other selective LTA4 hydrolase inhibitor is administered by pulsatile dosing or wherein the acebilustat is in a pulsatile release pharmaceutical composition. Pulsatile dosing encompasses administration or release of an active agent or drug after a pre-determined off-released period or lag time. As such, pulsatile dosing can include delivering a drug rapidly and completely after a period of no drug release. For example, the concentration of the active agent or drug in the plasma is allowed to drop below about 50% inhibition of the LTA4H activity or below about 50% of LTB4 generation before the next dose is administered. The acebilustat or other selective LTA4H inhibitor can also be administered in a controlled release, sustained release manner, and/or delayed release manner. The acebilustat or other selective LTA4H inhibitor can also be administered in a controlled release formulation, including, for example, a sustained release formulation and/or a delayed release formulation. “Controlled release” refers to a drug-containing formulation or unit dose form thereof from which release of the drug is not immediate, i.e., with a controlled release formulation, administration does not result in immediate release of all of the drug administered into an absorption pool. The term is used interchangeably with “nonimmediate release” as defined in Remington: The Science and Practice of Pharmacy, Nineteenth Ed. (Easton, Pa.: Mack Publishing Company, 1995). In general, controlled release formulations include sustained release and delayed release formulations. “Sustained release” and “extended release” means a drug formulation that provides for gradual release of a drug over an extended period of time, and typically, although not necessarily, results in substantially constant blood levels of a drug over an extended time period. “Delayed release” refers to a drug formulation that, following administration to a patient, provides a measurable time delay before drug is released from the formulation into the patient's body.

The treatment can include administration of the same dose and/or dosing regimen throughout the patient's treatment. The method can also include an initial dose(s) of the acebilustat or other selective LTA4H inhibitor followed by maintenance therapy comprising one or more maintenance doses, wherein the one or more maintenances doses are different from the initial dose. For example, the one or more maintenance doses can be lower than the initial dose and/or can be less frequent than the initial dosing regimen. In another example, the one or more maintenance doses can be higher than the initial dose and/or can be more frequent than the initial dosing regimen.

The treatment can be initiated (e.g., the initial dose can be administered), for example, immediately, or within one week, or one to three days after surgery. The treatment can also be initiated after surgical wound healing is complete, or immediately, or within one week, or one to three days of initiation of radiotherapy, or cancer treatment, or at a time point before the onset or diagnosis of stage 0 lymphedema. The treatment can also be initiated after the onset or diagnosis of stage 0 lymphedema or after the onset or diagnosis of clinically evident lymphedema.

The invention also encompasses methods of treating lymphedema using selective LTA4H inhibitors other than acebilustat. LTA4H is an epoxide hydrolase that generates LTB4 from LTA4. LTB4 is a pro-inflammatory mediator and thus inhibition of LTA4H activity inhibits the production of the pro-inflammatory LTB4. LTA4H also is an aminopeptidase and degrades the tripeptide Pro-Gly-Pro (PGP) which is a neutrophil chemoattractant (Low et al. (2017), Scientific Reports 7, 44449 (2017). https://doi.org/10.1038/srep44449; the contents of which are expressly incorporated by reference herein). The accumulation of PGP is associated with pro-inflammatory effects. Thus, LTA4H plays an important anti-inflammatory role in degrading PGP, which role is paradoxical to its pro-inflammatory epoxide hydrolase activity. The present invention is at least partially based on the discovery that lymphedema, including secondary lymphedema, can advantageously be treated using a selective LTA4H inhibitor such as acebilustat. A selective LTA4H inhibitor is an agent or compound that has more selectivity for LTA4H than other aminopeptidase enzymes and/or an agent that has more selectivity for the epoxide hydrolase activity of LTA4H than the aminopeptidase activity of LTA4H. In the context of LTA4 inhibitors, selectivity refers to potency of inhibition. For example, an LTA4H inhibitor is more selective for the epoxide hydrolase activity of LTA4H than the aminopeptidase activity of LTA4H when the IC₅₀ for epoxide hydrolase inhibition is lower than the IC₅₀ for aminopeptidase activity. In another example, an LTA4H inhibitor is more selective for inhibiting LTA4H than other aminopeptidases when the IC₅₀ for LTA4H inhibitor is lower than the IC₅₀ for other aminopeptidases. A selective LTA4H inhibitor that is more selective for the epoxide hydrolase activity of LTA4H than the aminopeptidase activity of LTA4H inhibits generation of LTB4 while having minimal effect on PGP degradation. Acebilustat is a selective LTA4H inhibitor that is more selective for the epoxide hydrolase activity of LTA4H than the aminopeptidase activity of LTA4H and is more selective for LTA4H inhibition than aminopeptidase inhibition. Specifically, acebilustat has a 2-fold preference for inhibiting epoxide hydrolase activity versus aminopeptidase activity (Bhatt et al. (2017), Seminars in Immunology 33:65-73; the contents of which are expressly incorporated by reference herein). Specifically, Bhatt et al. teaches that acebilustat shows 2-fold functional selectivity for LTA4H epoxide hydrolase (12 nM) versus LTA4H aminopeptidase (27 nM) and is highly selective for LTA4H versus other metalloenzymes. In contrast, some other LTA4H inhibitors, such as SC567461A (Searle/Pharmacia), DG-051 (DeCODE Pharmaceuticals), and JNJ-40929837 (Johnson and Johnson), have similar potencies at inhibiting LTB4 generation and PGP degradation and thus may have a pro-inflammatory effect by resulting in PGP accumulation. Such inhibitors are referred to herein as “non-selective LTA4H inhibitors.” Ubenimex is a non-selective LTA4H inhibitor and is a broad spectrum aminopeptidase inhibitor (Bhatt et al. (2017); Inoi et al. (1995), Anticancer Res. 15 (5B): 2081-2087; the contents of which are expressly incorporated by reference herein).

The IC₅₀ of LTA4H epoxide hydrolase activity can be measured using methods known in the art, for example, using the LTA4 hydrolase homogenous time resolved fluorescence assay which is described, for example, in U.S. Pat. Nos. 7,737,145 and 10,202,362; the contents of which are expressly incorporated by reference herein. The hydrolase-homogeneous time resolved fluorescence assay is a two-step assay that measures the hydrolysis of LTA₄ to LTB₄ by analyzing the amount of LTB₄ produced. The first step involves the enzymatic conversion of LTA₄ to LTB₄ and the second step involves the quantification of the LTB4 formed with a homogeneous time resolved fluorescence assay. LTA4H epoxide hydrolase activity can also be measured in a whole blood assay, for example, using human whole blood, using the method described in Penning, T. D. et al., J. Med. Chem. (2000), 43 (4): 721-735 and U.S. Pat. No. 7,737,145; the contents of each of which are expressly incorporated by reference herein. In the whole blood assay, the inhibitor compounds are tested for their ability to inhibit LTB4 release upon stimulation with calcium ionophore and the LTB4 levels in supernatants are measured by ELISA. LTA4H epoxide hydrolase activity can also be measured according to the method described in Low et al. (2017). The IC₅₀ for LTA4H aminopeptidase activity can be measured using methods known in the art, for example, using the method described in Kull et al., The Journal of Biological Chemistry 274 (49): 34683-34690, the method described in U.S. Pat. No. 10,202,362, and/or the method described in Low et al. (2017); the contents of each of which are expressly incorporated by reference herein. Other methods for measuring LTA4H epoxide hydrolase activity and/or LTA4H peptidase activity are described, for example, in Askonas, L. J., et al., The Journal of Pharmacology and Experimental Therapeutics 2002, 300 (2): 577-582; Penning, T. D., J. Med. Chem. 2000, 43 (4): 721-735; Kull, F. et al., The Journal of Biological Chemistry 1999, 274 (49): 34683-34690; the contents of which are expressly incorporated by reference herein.

The present invention encompasses a method of treating lymphedema comprising administering to a patient in need thereof an effective amount of a selective LTA4H inhibitor. The selective LTA4H inhibitor can, for example, have at least about 1.5 times or at least about 2 times more selectivity for the epoxide hydrolase activity of LTA4H versus the aminopeptidase activity of LTA4H. The selective LTA4H inhibitor can additionally or alternatively, have at least about 1.5 times, at least about 2 times, at least about 2.5 times at least about 3 times, at least about 5 times, or at least about 10 times more selectivity for LTA4H than other aminopeptidases. In yet another embodiment, the LTA4H inhibitor is at least about 2 times more selective for LTA4H than other aminopeptidases and more than about 1.5 times more selective for epoxide hydrolase activity than aminopeptidase activity of LTA4H. The LTA4H inhibitor can also be at least about 2.5 times, at least about 3 times, at least about 5 times, or at least about 10 times more selective for epoxide hydrolase activity than aminopeptidase activity of LTA4H. In yet a further embodiment, the LTA4H inhibitor is at least about 2 times more selective for LTA4H than other aminopeptidases and more than about 2 times more selective for epoxide hydrolase activity than aminopeptidase activity of LTA4H. The selective LTA4H inhibitors include, for example, acebilustat. Other selective LTA4H inhibitors have been described in Low et al. and include compounds with a resveratrol core as described therein. Non-limiting examples of LTA4H inhibitors include, for example, cis-resveratrol, trans-resveratrol, isoflavone daidzein, and 7,8,4′-trihydroxyisoflavone. Additional, non-limiting examples of LTA4H inhibitors are shown in the Tables below:

TABLE 1

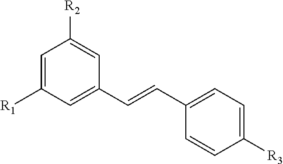

| | | | | | Amino- |
|---|---|---|---|---|---|
| | | | | EH | peptidase |
| | | | | IC50 ± SEM | IC50 ± SEM |
| Cmpd No. | R1 | R2 | R3 | (uM) | (uM) |
| 1 | OH | OH | OH | 32.2 ± 5.9 | >100 |
| 2 | OH | OH | OH | 14.3 ± 2.1 | 85.3 ± 33.9 |
| (Z-isomer) | | | | | |
| 4 | OCOMe | OCOMe | OCOMe | 38.6 ± 5.9 | >100 |
| 5 | OMe | OH | OH | 3.9 ± 0.4 | 99.5 ± 22.6 |

TABLE 2

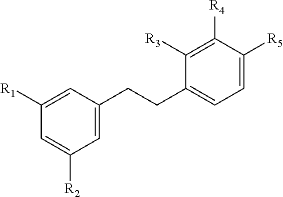

| Cmpd | | | | | | EH | Aminopeptidase |
|---|---|---|---|---|---|---|---|
| No. | R1 | R2 | R3 | R4 | R5 | IC50 ± SEM (uM) | IC50 ± SEM (uM) |
| 6 | OMe | OH | H | H | OH | 6.4 ± 0.7 | >100 |
| 7 | OEt | OH | H | H | OH | 3.2 ± 0.7 | >100 |
| 8 | OMe | OH | H | OH | H | 1.7 ± 0.25 | >100 |
| 9 | OMe | OH | OH | H | H | 0.5 ± 0.04 | >100 |

TABLE 3

| | |
|---|---|
| | Compound 10 |
| 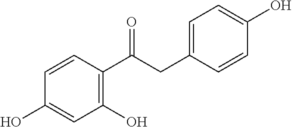 [SMILES: O=C(Cc1ccc(O)cc1)c1ccc(O)cc1O] | |

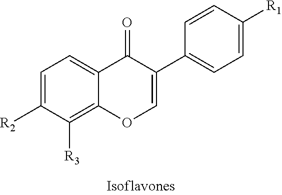

| | | | | Epoxide Hydrolase | Aminopeptidase |
|---|---|---|---|---|---|
| Cmpd. No. | R₁ | R₂ | R₃ | IC50 ± SEM (uM) | IC50 ± SEM (uM) |
| 10 | | | | 34.24 ± 4.7 | >100 |
| 11 | OH | OH | H | 15.0 ± 2.3 | >100 |
| 12 | OH | OR | OH | 7.3 ± 0.8 | 62.7 ± 13.3 |
| 13 | H | OH | H | 48.2 ± 14.6 | >100 |
| 14 | OH | O—CH₂—O | | 20.6 ± 24 | 20.6 ± 3.3 |
| 15 | OH | Me | OH | 1.2 ± 0.21 | 69.2 ± 10.9 |

In some embodiments, an effective dose of acebilustat is administered to an individual having lymphedema, including without limitation, established lymphedema, for a period of time sufficient to decrease or reverse tissue pathology of the affected (lymphadematous) tissue relative to an untreated control group. Treatment can be continued as required for maintenance of the therapeutic benefit: where required, maintenance therapy can be maintained at the same dosage and schedule as previous treatment or may be achieved by transitioning to an alternative maintenance schedule, e.g., at a lower dose, less frequent dose, and the like. In some embodiments, the treating physician can determine that the treatment is efficacious by verifying a change in the architecture of the affected tissue. The tissue can be assayed by any number of means as described herein to verify therapeutic benefit, if visual inspection alone is insufficient. A convenient measure of efficacy in some applications is dermal thickness, although those of skill in the art will understand upon contemplation of this disclosure that various indicia can be used to monitor treatment and determine efficacy. Additional methods of determining if the treatment is efficacious include lymphoscintigraphy and indocyanine green (ICG) lymphography.

In some embodiments, an effective dose of acebilustat or other selective LTA4H inhibitor is provided to an individual susceptible to lymphedema, including without limitations individuals that have undergone surgery or radiation for cancer. In further aspects, the lymphedema is secondary, or acquired lymphedema.

In other embodiments, the invention is directed to a method of treating lymphedema and the patient has lymphedema (stage 0 to 3) as a result of cancer therapy, e.g., surgery or radiotherapy or other therapy damaging to the lymphatic system. In some embodiments, the individual has been treated with surgery, typically as a result of cancer diagnosis and treatment but other surgeries affecting the lymph nodes can cause lymphedema treatable in accordance with the invention. In such embodiments, treatment with acebilustat or other selective LTA4H inhibitor can be initiated immediately following surgical wound healing, or can be initiated at a time following surgical wound healing, including after some or even substantial wound healing has occurred, e.g., 3-14 days after surgery, but before a patient has been diagnosed as having stage 0 lymphedema. In yet additional aspects, the patient has been treated with radiotherapy, which itself may follow surgery for cancer therapy. In such embodiments, treatment with acebilustat or other selective LTA4H inhibitor can be initiated immediately during radiotherapy, following radiotherapy, or may commence at a time following radiotherapy, including after some or even substantial wound healing has occurred, as above, but before a patient has been diagnosed as having stage 0 lymphedema.

The invention includes a method of treating upper limb extremity lymphedema in patients after cancer surgery and/or radiation therapy. The invention additionally includes a method of treating upper limb extremity lymphedema in a patient after breast cancer surgery and/or radiation therapy. Arm or upper limb lymphedema is caused by interruption of the axillary lymphatic system by surgery or radiation therapy, resulting in accumulation of fluid in subcutaneous tissue in the arm.

The invention also includes a method of preventing or inhibiting progression of lymphedema in an individual to be treated has, or that has been, or is being treated for cancer but has not yet developed lymphedema. The invention can also be practiced in other prophylactic modes, including after successful treatment. Some patients can be treated in accordance with the invention and obtain complete or significantly complete recovery and not benefit from further treatment. Other patients, however, can benefit from continued administration of acebilustat or other selective LTA4H inhibitor after treatment has been successful to prevent recurrence of the disease. Thus, for prophylactic purposes of lymphedema prevention, following treatment by the methods of the invention, the architecture of the skin of the affected patient, e.g., skin of the extremities, remains with a substantially normal architecture consistent with successful treatment. Following prophylactic treatment with the methods of the invention, the volume of tissue, e.g., upper extremities, lower extremities, etc. should be stable over time, relative to a control group in the absence of treatment. The time period in which to see treatment benefit can be about 1 week, about 2 weeks, about 3 weeks, about 1 month, about 2 months, about 3 months, about 4 months, or more. In some embodiments, the volume and/or structure of the affected or at risk tissue, i.e., the lymphadematous tissue, is measured or otherwise assayed or assessed at various points over the time period, e.g., at least at the beginning and some designated endpoint for testing. In some embodiments, the architecture of the affected tissue is assayed by dermal thickness measurements or by histological assessment; absence of lymphedema can be ascertained by serial measurement of limb bioimpedance. The effect on the lymphatic system can also be assessed using lymphoscintigraphy and/or indocyanine green lymphography, for example. In some embodiments, the architecture of the affected tissue after treatment resembles or more closely resembles the architecture of unaffected tissue.

Acebilustat can be administered to a patient on top of their current treatment regime, or on top of the standard of care. The standard of care for the treatment of lymphedema includes, but is not limited to, diuretics, antibiotics, exercise, manual lymph drainage, compression bandages, as well as compression garments, for example. The methods of the invention can also include administration of a therapeutically effective amount of at least one additional active agent other than a LTA4H inhibitor. The additional agent can, for example, be selected from the group consisting of a selective COX-1 inhibitor, a selective COX-2 inhibitor, a non-selective COX-1/COX-2 inhibitor, a coumarin, an anti-histamine, montelukast and other related leukotriene inhibitors, an anti-fibrotic compound, a diuretic, a statin, an mTOR inhibitor, and pirfenidone. In certain aspects, the additional agent can, for example, be selected from the group consisting of a selective COX-1 inhibitor, a selective COX-2 inhibitor, a non-selective COX-1/COX-2 inhibitor, a coumarin, an anti-histamine, an anti-fibrotic compound, a diuretic, a statin, an mTOR inhibitor, and pirfenidone. Non-limiting examples of non-selective COX-1/COX-2 inhibitors include salicylic acid derivatives including aspirin, sodium salicylate, choline magnesium trisalicylate, salsalate, diflunisal, sulfasalazine and olsalazine; para-aminophenol derivatives such as acetaminophen, indole and indene acetic acids such as indomethacin and sulindac, heteroaryl acetic acids including tolmetin, diclofenac and keterolac, arylpropionic acids including ibuprofen, naproxen, flurbiprofen, ketoprofen, fenoprofen and oxaprozin, anthranilic acids (fenamates) including mefanamic acid and meclofenamic acid, enolic acids including oxicams such as piroxicam and meloxicam and alkanones such as nabumetone, as well as pharmaceutically effective esters, salts, isomers, conjugates and prodrugs thereof. In yet additional aspects, the non-selective COX-1/COX-2 inhibitor is a propionic acid derivative, such as ketoprofen. Selective COX-2 inhibitors include, for example, celecoxib. Non-limiting examples of diuretics are furosemide, torasemide and hydrochlorothiazide, recombinant angiotensin converting enzyme-2 and acetylsalicylic acid. Statins include, for example, atorvastatin, fluvastatin, lovastatin, pitavastatin, pravastatin, rosuvastatin, simvastatin, and/or ezetimibe/simvastatin combination, mTOR inhibitors include, for example, rapamycin, everolimus, and sirolimus, and rapalog. Anti-fibrotic agents include the following non-limiting examples: pirfenidone, Transforming Growth Factor beta (TGFβ) inhibitors such as galunisertib, and Connective Tissue Growth Factor (CTGF) inhibitors such as FG-3019. The additional active agent can also be any other agent used in the treatment of lymphedema and/or pro-lymphangiogenic drugs like retinoic acids (including, for example, 9-cis retinoic acid). In another example, the additional active agent is an agent, e.g., a biologic or a small molecule that that targets and/or inhibits an anti-lymphangiogenic growth factor or cytokine including, but not limited to, anti-TGF-β1 antibodies, anti-IFN-γ antibodies, anti-IL-4 antibodies and anti-IL-13 antibodies. For example, the additional agent can be an antibody (e.g., a monoclonal antibody) that targets and/or inhibits an anti-lymphangiogenic growth factor or cytokine including, but not limited to, anti-TGF-β1 antibodies, anti-IFN-γ antibodies, anti-IL-4 antibodies and anti-IL-13 antibodies. The antibody can be one that targets one or more anti-lymphangiogenic growth factors or cytokines including, but not limited to, dupilumab (a monoclonal antibody that inhibits IL-4 and IL-13), and pascolizumab (a humanized anti-IL-4 monoclonal antibody). The additional agent can also be pitrakinra, a human recombinant protein that is an antagonist of IL-4 and IL-13.

Also, the invention provides a pharmaceutical formulation comprising acebilustat or other selective LTA4H inhibitor and an additional active agent, such as a selective COX-1 inhibitor, a selective COX-2 inhibitor, a non-selective COX-1/COX-2 inhibitor, a coumarin, an anti-histamine, montelukast and related leukotriene modifiers, an anti-fibrotic compound, a diuretic, a statin, an mTOR inhibitor, and pirfenidone. In another aspect, the pharmaceutical formulation comprises acebilustat or other selective LTA4H inhibitor and an additional active agent selected from a selective COX-1 inhibitor, a selective COX-2 inhibitor, a non-selective COX-1/COX-2 inhibitor, a coumarin, an anti-histamine, an anti-fibrotic compound, a diuretic, a statin, an mTOR inhibitor, and pirfenidone. The invention also includes a method of treating lymphedema, where the method includes a combination therapy in which a patient in need of treatment is administered an effective dose of acebilustat or other selective LTA4H inhibitor in combination with one or more drugs or other therapies approved or used in the treatment of lymphedema and/or pro-lymphangiogenic drugs like retinoic acids (including, for example, 9-cis retinoic acid). The invention additionally includes a method of treating lymphedema, wherein the method includes a combination therapy in which a patient in need of treatment is administered an effective dose of acebilustat or other selective LTA4H inhibitor in combination with one or more additional active agents (e.g., a biologic or a small molecule) that targets and/or inhibits an anti-lymphangiogenic growth factor or cytokine including, but not limited to, anti-TGF-β1 antibodies, anti-IFN-γ antibodies, anti-IL-4 antibodies and anti-IL-13 antibodies. For example, the additional agent can be an antibody (e.g., a monoclonal antibody) that targets and/or inhibits an anti-lymphangiogenic growth factor or cytokine including, but not limited to, anti-TGF-β1 antibodies, anti-IFN-γ antibodies, anti-IL-4 antibodies and anti-IL-13 antibodies. The antibody can be one that targets one or more anti-lymphangiogenic growth factors or cytokines including, but not limited to, dupilumab (a monoclonal antibody that inhibits IL-4 and IL-13), and pascolizumab (a humanized anti-IL-4 monoclonal antibody). The additional agent can also be pitrakinra, a human recombinant protein that is an antagonist of IL-4 and IL-13.

In yet another aspect, the acebilustat or other selective LTA4H inhibitor is administered in combination with one or more other drugs useful in preventing or treating lymphedema. In some of these embodiments, the acebilustat or other selective LTA4H inhibitor is administered with a different LTB4 inhibitor. For example, the different LTB4 inhibitor can be of the BLT1/BLT2 antagonist classes of LTB4 inhibitors.

It is to be understood that when the acebilustat or other selective LTA4H inhibitor is co-administered with the at least one additional active agent (such as a selective COX-1 inhibitor, a selective COX-2 inhibitor, a non-selective COX-1/COX-2 inhibitor, a coumarin, an anti-histamine, montelukast and related leukotriene modifiers, an anti-fibrotic compound, a diuretic, a statin an mTOR inhibitor, and pirfenidone, and/or other therapeutic agent such as a retinoic acid, and/or agent that targets one or more anti-lymphangiogenic growth factors or cytokines), the acebilustat or other selective LTA4H inhibitor can be administered simultaneously with, prior to, or after administration of one or more additional active agents. Such combination therapy includes administration of a single pharmaceutical dosage formulation which contains the acebilustat or other selective LTA4H inhibitor and the one or more additional active agents, as well as administration of the acebilustat and each active agent in its own separate pharmaceutical dosage formulation.

The acebilustat or other selective LTA4H inhibitor can continue to be administered if the therapy is determined to be efficacious. The methods can comprise maintaining, tapering, reducing, or stopping the administered amount of the acebilustat or other selective LTA4H inhibitor in the therapy if the therapy is determined to be efficacious. The methods can comprise increasing the administered amount of the acebilustat or other selective LTA4H inhibitor in the therapy if it is determined not to be efficacious or likely to be more efficacious if dosing is increased in daily amount or via a change in the administration schedule. Alternatively, the methods can comprise stopping therapy if it is determined not to be efficacious.

Treatment, for example, can commence at any time after the onset of stage 0 lymphedema, e.g., where treatment stabilizes or reverses patient condition to a non-symptomatic state. Treatment with the methods of the invention can, for example, commence following onset of any clinically evident lymphedema, such as stage 1. Treatment with the methods of the invention can also, for example, commence following onset of stage 2 lymphedema. Treatment with the methods of the invention can commence following onset of stage 3 lymphedema. Treatment can also commence before the onset of stage 0 lymphedema but after surgery, radiotherapy or other medical intervention that increases the risk of developing lymphedema.

The swelling that can accompany disease progression can be unilateral or bilateral, and may worsen when the weather is warm, before menstruation occurs, following physical exertion, and/or after the limb remains for a long time in a dependent position. It can affect any part of a limb (isolated proximal or distal) or the entire extremity, or the face, head and neck, trunk, breast or genitalia; it can restrict range of motion. Disability and emotional distress can be significant, especially when lymphedema results from medical or surgical treatment. Skin changes are common and include hyperkeratosis, hyperpigmentation, lichenification, verrucae, papillomas, and fungal infections. The methods of the invention include methods to treat any and all of these conditions and symptoms, including but not limited to by administration of acebilustat as described herein.

Lymphangitis or cellulitis can develop, for example, when bacteria traverse the skin barrier, which is impaired in lymphedema. Cellulitis in lymphedema may be characterized by only very subtle changes in the limb, and can be difficult to diagnose or eradicate. Lymphangitis is frequently streptococcal, causing erysipelas; sometimes it is staphylococcal. The affected limb becomes red and feels hot; red streaks may extend proximally from the point of entry, and lymphadenopathy may develop. Rarely, the skin breaks down. Rarely, long-standing lymphedema leads to lymphangiosarcoma (Stewart-Treves syndrome), usually in postmastectomy patients and in patients with filariasis. The methods of the invention include methods to treat any and all of these conditions and symptoms, including but not limited to by administration of acebilustat as described herein.

Without treatment, cellular overgrowth, adipose deposition and fibrosis promote the progressive anatomic distortion and loss of function of the affected areas. Additionally, impaired trafficking of antigen-presenting cells in lymph hampers local immune surveillance of the lymphedematous region(s) to the draining lymph nodes. Thus, there is chronic inflammation, infection, and hardening of the skin that, in turn, results in further lymph vessel damage and distortion of the shape of the affected body parts. Moreover, there is a high degree of dysfunction due to physical factors such as a decrease in joint mobility causing reduced amplitude of movements, increased extremity weight, increased pain, and impaired ability to perform day-to-day tasks. The methods of the invention include methods to treat any and all of these conditions and symptoms, including but not limited to by administration of acebilustat or other selective LTA4H inhibitor, as described herein.

Pathological skin changes associated with lymphedema include an increase in cellularity of layers of the skin, accumulation of glycoproteins, loss of elasticity, and subdermal increase in adipose layer. The methods of the invention include methods to treat any and all of these conditions and symptoms, including, but not limited to, by administration of acebilustat or other selective LTA4H inhibitor as described herein.

Those of skill will thus appreciate that the methods of the invention are applicable to the treatment and prevention of lymphedema including its signs and symptoms such as those associated with the following clinical indicia of lymphedema. A number of clinical indicia can be used to diagnose lymphedema and to monitor the effectiveness of therapy, including treatment with the compositions and methods of the present invention. The invention provides methods of determining efficacy of a lymphedema treatment in a subject in need thereof by (a) measuring an endpoint of a clinical indication in a patient, where the endpoint is measured after treatment has started, (b) comparing the endpoint of the clinical indication to a baseline or reference, where the baseline or reference is measured in the same subject or a similar subject population before treatment is begun, and (c) determining the efficacy of the lymphedema treatment based on the comparison step.

Analysis of clinical indicia may include measurement of dermal thickness; change of lymphedema volume of leg/arm/hand; change of stagnation of fluid at level of shoulder/trunk; change of extracellular fluid in arm; change of thickness and reflectivity of cutis and subcutis of arm/shoulder/trunk; change of elasticity of skin and subcutaneous tissue of arm; change of lymphatic architecture and function; change of venous circulation in arm/trunk; number of episodes of cellulitis.

When imaging is used to diagnose lymphedema or assess disease state or progression, the most common modality for diagnosis is indirect radionuclide lymphoscintigraphy. This procedure requires subcutaneous injection of an appropriate radiolabeled tracer, for example ^{99m}Tc-antimony sulfide colloid or ^{99m}Tc-labeled human serum albumin. Criteria for the diagnosis of lymphatic dysfunction include: (1) delayed, asymmetric or absent visualization of regional lymph nodes; (2) asymmetric visualization of lymphatic channels; (3) collateral lymphatic channels; (4) dermal backflow; (5) interrupted vascular structures; and (6) visualization of the lymph nodes of the deep lymphatic system. The presence of “dermal back-flow” is considered abnormal. It is interpreted to represent the extravasation of lymph fluid from the lymphatics into the interstitium as a result of lymphatic and/or venous hypertension. Beyond lymphoscintigraphy, magnetic resonance imaging and computerized axial tomography have clinical utility. These imaging techniques permit objective documentation of the structural changes caused by lymphedema. Recent advances in the magnetic resonance approach have improved the visualization of lymphatic vascular anomalies in both nonenhanced and contrast-enhanced applications (see, for example, Pankaj et al. (2013) World J Surg Oncol. 2013; 11:237). As an alternative, bioelectrical impedance has been used to detect and monitor upper limb lymphedema (see Ridner et al. (2009) Lymphat Res Biol. 7 (1): 11-15), which uses characteristics of frequency-dependent current flow to quantify changes in extracellular fluid. In various embodiments, such technology is used to monitor the progress of therapy of a patient treated in accordance with the invention or to identify a patient that may benefit from such treatment. Imaging can also include indocyanine green (ICG) fluorescent lymphography as described, for example in Suami et al., BMC Cancer 19, 985 (2019). https://doi.org/10.1186/s12885-019-6192-1, the contents of which are expressly incorporated by reference herein.

The effectiveness of treatment by the methods of the invention will be evidenced by improvement in disease symptoms and pathology. Individuals being treated and medical practitioners may choose to evaluate success, monitor the course of treatment, adjust dosage and timing, etc. by any convenient indicia.

In some embodiments of the invention, e.g., treatment of patients with established disease, improvements in the architecture of the skin provide a convenient method for assessing treatment success. For example, dermal thickness reflects the architectural changes in lymphedema. See, for example, Mellor et al., Breast J 2004; 10:496-503; Hacard et al. Skin Res Technol 2014; 20:274-81, each herein specifically incorporated by reference. The invention encompasses methods that reduce dermal thickening associated with lymphedema. Dermal thickness can be, for example, measured with factory calibrated skinfold calipers, such as Lange skinfold calipers, Model EQ0014921. In additional aspects, dermal thickness or the decrease in dermal thickening is measured using ultrasound. In some embodiments, a treatment provided herein is efficacious if, after a period of time from the onset of treatment (e.g., 2 weeks, 1 month, 2 months, 3 months, 4 months, 5 months, 6 months or longer), there is a decrease in dermal thickness of at least one affected region (e.g., limb) as compared to the dermal thickness of the at least one affected region prior to the onset of treatment. The decrease in dermal thickness observed with successful treatment can be a decrease of at least about 1 mm, at least about 2.5 mm, at least about 5 mm, at least about 7.5 mm, at least about 10 mm, and may be at least about 12.5 mm, at least about 15 mm, or more, as compared to that before the treatment is initiated (referred to herein as “baseline”) or at an earlier point in the treatment regimen. In yet additional aspects, the dermal thickening can be decreased by at least about 5%, about 10%, about 20%, or more, as compared to that before the treatment is initiated (referred to herein as “baseline”) or at an earlier point in the treatment regimen Additional measurements for determining a correction of pathologies of skin architecture may include, for example, DEXA scanning, direct biopsy, visual inspection, etc. Dermal thickness and architecture, e.g., presence of hyperkeratosis, dermal collagen, and adipose deposition in an affected limb can be monitored.

In some embodiments, a change in the volume of the affected limb is measured as a measure of treatment success, i.e., the volume declines with successful treatment. Volume can be measured by any of a number of methods in the art, e.g., circumferential measurements, water displacement volumetry, etc. For example, an assessor may use a standardized tape measure for circumference measurements taken every 2-6 cm, and calculating the volume by, for example, the truncated cone method. Successful treatment can reduce, or decrease, the volume of lymphedematous body parts (both the fluid and tissue components). In some instances, volume is decreased 2-fold or more after treatment, i.e., as compared to the volume before treatment, for example, 2-fold or more, 3-fold or more, 4-fold or more, sometimes 5-fold or more, 10-fold or more, 15-fold or more, in some instances 20-fold or more, 50 fold- or more, etc. In other words, the volume is decreased by about 50 milliliters or more, 100 milliliters or more, 200 milliliters or more, 300 milliliters or more, 400 milliliters or more, 500 milliliters or more. In some instances, the volume is restored to normal volume, i.e., the volume prior to the onset of the lymphedema, e.g., the volume of the unaffected bilateral tissue.

In some embodiments, the level of serum LTB4 can be used for diagnosis, or selecting, or stratifying patients for therapy, or for monitoring efficacy. A reference value of normal LTB4 can be (depending on assay type and format and individual laboratory practices) up to about 50 μg/mL or greater, up to about 100 μg/mL or greater, up to about 200 μg/mL or greater, up to about 250 μg/mL or greater, up to about 300 μg/mL. Individuals with lymphedema may have elevated baseline levels of serum LTB4, where the serum levels are up to about 1000 μg/ml or greater, and can range from about 500 μg/ml to about 1500 μg/ml. In certain aspects, the level of serum LTB4 in a sample obtained from a patient is measured and if it above a predetermined threshold level, the patient is administered acebilustat or other selective LTA4H inhibitor. The treatment provided in the invention can be determined to be efficacious if, after treatment has started, the endpoint LTB4 level of the subject decreases from the baseline LTB4 level. In other embodiments, the treatment provided in the invention is efficacious if, after treatment has started, the endpoint LTB4 level is lower than the baseline LTB4 level by 2-fold or more, 3-fold or more, 4-fold or more, 5-fold or more, 10-fold or more, 15-fold or more. For example, in some embodiments a higher or elevated level of LTB4 in a blood sample, of at least 3-fold higher, at least 4-fold higher, at least 5-fold, at least about 10-fold higher, at least about 15-fold higher than the control value indicates that the patient is in need of treatment using a therapy of the invention or that the patient is likely to respond to a therapy of the invention. Standard methods for assessing LTB4 levels are utilized. The method further comprises administering an effective amount of acebilustat or other selective LTA4H inhibitors to a patient determined to be likely to benefit from, in need of, or likely to respond to, a therapy of the invention, thereby treating or preventing lymphedema in the patient.

The invention also includes a method of reducing the incidence of soft-tissue infection, e.g., cellulitis, in lymphedema patients comprising administering acebilustat or other selective LTA4H inhibitor. Skin infections or cellulitis are common complications of lymphedema. The methods described herein can be used to reduce the risk of cellulitis in lymphedema comprising administering acebilustat or other selective LTA4H inhibitor to a patient in need thereof.

As used therein, a “therapeutically effective amount” or an “effective amount” refers to that amount of a compound or drug that, when administered to a mammal, preferably a human, is sufficient to effect treatment, as defined below, of a disease or condition of interest in the mammal, preferably a human. The amount of a compound of the invention which constitutes a “therapeutically effective amount” or an “effective amount” will vary depending on, for example, the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy, but it can be determined routinely by one of ordinary skill in the art having regard to his own knowledge and to this disclosure. In certain aspects, an effective amount or a therapeutically effective amount of acebilustat or other selective LTA4H inhibitor is an amount that inhibits LTA4H or inhibits LTB4, and/or that treats or inhibits or decreases the severity of the disease and/or reduces or reduces dermal thickening and/or or improves skin turgor, histology and/or function or increases lymphatic flow and/or improves vascular function.

“Treating” or “treatment” as used herein covers the treatment of lymphedema, preferably a human, and includes, for example: (i) inhibiting or decreasing the severity of the disease or condition, or one or more symptoms thereof, i.e., arresting or slowing development or progression of the disease or condition and/or reducing dermal thickening and/or or improving skin turgor, histology and/or function and/or increasing lymphatic flow, and/or ameliorating or decreasing one or more symptoms; (ii) relieving the disease or condition, i.e., causing regression of the disease or condition, or one more symptoms thereof; and/or (iii) stabilizing the disease or condition. “Treating” or “treatment” can include decreasing the risk of progression of lymphedema.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

A “pharmaceutical composition” refers to a formulation of a compound described herein, for example, acebilustat or other selective LTA4H inhibitor and/or an additional therapeutic agent, and a medium generally accepted in the art for the delivery of the biologically active compound to mammals, for example, humans. Such a medium includes all pharmaceutically acceptable carriers, diluents or excipients.

“Optional” or “optionally” means that the subsequently described event or circumstances may or may not occur, and that the description includes instances where said event or circumstance occurs and instances in which it does not.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which, for example, has been approved by the United States Food and Drug Administration as being acceptable for use in humans or domestic animals.

Administration of the compounds or drugs described herein encompasses administration of a pharmaceutically acceptable salt of said compound or drug, for example, administration of a pharmaceutically acceptable salt of acebilustat or other selective LTA4H inhibitor. Administration of the compounds or drugs as described herein (such as acebilustat or other additional therapeutic agent), or their pharmaceutically acceptable salts, in pure form or in an appropriate pharmaceutical composition, can be carried out via any of the accepted modes of administration of agents for serving similar utilities. As described herein, an exemplary mode of administration for acebilustat is oral administration. The pharmaceutical compositions described herein can be prepared by combining a compound or drug with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols.

Routes of administering such pharmaceutical compositions include, without limitation, oral, topical, transdermal, inhalation, parenteral, sublingual, rectal, vaginal, and intranasal. The term parenteral as used herein includes subcutaneous injections, intravenous, intramuscular, intrasternal injection or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see The Science and Practice of Pharmacy, 20^{th} Edition (Philadelphia College of Pharmacy and Science, 2000). The composition to be administered will, in any event, contain a therapeutically effective amount of the compound or drug, or a pharmaceutically acceptable salt thereof, for treatment of a disease or condition of interest in accordance with the teachings of this invention.

As described, above, the pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, and intrathecal methods. Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Formulations suitable for enteral administration, such as, for example, administration topically (e.g., as solutions, lotions, creams, paste, emulsions, suspensions, etc.), orally, rectally, vaginally, or by inhalation, include capsules, liquid solutions, emulsions, suspensions, and elixirs. For example, if prepared for topical applications, the compositions may comprise a biocompatible organic solvent, e.g., an isopropyl ester such as isopropyl myristate and isopropyl palmitate; a polar lipid, e.g., lecithin, phosphatidylcholine, etc., a surfactant, e.g., docusate sodium, docusate sodium benzoate, docusate calcium, tween 80, polysorbate 80; water; and/or urea (present at a concentration of about 5 to 20% by mass of the final composition). In some instances, a topical formulation will comprise an enhancer for skin penetration, such as SEPA 09. Examples of topical formulations may be found in, e.g., U.S. Pat. Nos. 5,654,337, 5,093,133, 5,210,099, 3,957,971, 5,016,652, the complete disclosures of which are incorporated herein by reference.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

A pharmaceutical composition can be in the form of a solid or liquid. In one aspect, the carrier(s) are particulate, so that the compositions are, for example, in tablet or powder form. In one aspect, the composition can be an encapsulated powder or granular form. In another aspect, an encapsulated powder or granular formulation can be opened and sprinkled in food or administered by gastric intubation. The carrier(s) can be liquid, with the compositions being, for example, an oral syrup, injectable liquid or an aerosol, which is useful in, for example, inhalatory administration. When intended for oral administration, the pharmaceutical composition can be in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition can be in the form of a liquid, for example, an elixir, syrup, solution, emulsion or suspension. The liquid can be for oral administration or for delivery by injection, as two examples. When intended for oral administration, a composition can contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the invention, whether solutions, suspensions or other like form, can include one or more of the following adjuvants: sterile diluents such as water for injection, saline solution, preferably physiological saline, Ringer's solution, isotonic sodium chloride or physiological saline, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

The invention is illustrated by the following non-limiting examples.

### Example 1: A Pilot Placebo-Controlled Trial of Acebilustat (CTX-4430) for the Treatment of Human Upper Extremity Lymphedema

### 1. Study Agent

Acebilustat, also known by the code name CTX-4430, is a novel synthetic small molecule leukotriene A4 hydrolase (LTA4H) inhibitor being developed for the treatment of inflammatory conditions. The chemical name of CTX-4430 is 4-[[(1S,4S)-5-[[4-[4-(2-oxazolyl)phenoxy]phenyl]methyl]-2,5-diazabicyclo[2.2.1]hept-2-yl]methyl]-benzoic acid. It is a chiral molecule that is manufactured as a single isomer. Investigational product is manufactured as a powder blend in size 0, white, opaque, hard gelatin capsules and supplied in white high-density polyethylene (HDPE) bottles in quantities of 30-36 capsules per unit. Investigational product is stable at controlled room temperature (15 to 25° C./59 to 77° F.) for at least 4 years when protected from exposure to ultraviolet (UV) light.

Mechanistically, Acebilustat is a potent and selective inhibitor of LTA4H-mediated production of the potent inflammatory mediator Leukotriene B4 (LTB4) with an IC₅₀ (concentration resulting in 50% inhibitory effect) of 0.013 μM in vitro and an IC₅₀ of 0.064 μM in ex vivo stimulated human whole blood. Pharmacodynamic (PD) studies in animals and humans treated orally with Acebilustat demonstrate a strong correlation between plasma concentrations of Acebilustat and the percent inhibition of LTB4 production in ex vivo stimulated whole blood. In human clinical PD studies, the 50% effective concentration (EC₅₀) of Acebilustat for inhibition of LTB4 production in blood was found to be 93 ng/mL (0.19 μM). In addition to decreasing LTB4 production, a potential additional pharmacologic benefit of LTA4H inhibition is increasing concentration of the anti-inflammatory mediator lipoxin A4 (LXA4) via a shunting mechanism, although this effect has not been conclusively shown to occur in humans treated with Acebilustat²⁰.

The nonclinical testing of Acebilustat included a comprehensive program of pharmacology, pharmacokinetic (PK), metabolism, and toxicology studies conducted to support human clinical studies. Pharmacologically, Acebilustat reduces production of leukotriene B4 (LTB4). LTB4 is a key mediator of inflammation that acts by inducing migration and activation of neutrophils and other immune cells. Acebilustat is rapidly absorbed and broadly distributed to tissues and then eliminated primarily via liver-bile-feces, with less than 3% of total drug excreted via kidneys. Mass balance studies in animals showed some potential for accumulation and retention of Acebilustat in pigmented tissues, notably the uveal tract, with no evidence for ophthalmologic changes in long term safety studies. In vitro studies have suggested potential for drug interactions with substrates of P-glycoprotein (P-gp) and multidrug and toxin extrusion (MATE)-2K, which should be taken into consideration for future clinical studies until definitive clinical drug interaction studies are conducted. Preclinical studies showed no potential for phototoxic effects. The dose limiting toxicity observed in long-term animal safety studies across species was failure to gain weight associated with reduced food consumption. Nonclinical toxicology studies support chronic administration of Acebilustat in doses up to 200 mg/day to humans aged 2 years and older.

Not including the ongoing clinical study for treatment of mild COVID-19, 327 adult subjects (including 6 subjects who received a single dose of [14C]-Acebilustat) have received at least one dose of Acebilustat to date in a total of six completed clinical studies sponsored by Celtaxsys Inc (IND owner prior to Celltaxis LLC); of these, 112 subjects with cystic fibrosis (CF) were treated for 48 weeks. These studies include a single- and multiple-dose first-in-human study for up to 14 days in healthy volunteers (CTX-4430-HV-001, NCT01748838), a multiple-dose study for 15 days in adult CF patients (CTX-4430-CF-001, NCT01944735), a multiple-dose drug-drug interaction study in healthy volunteers for 7 days to assess potential for induction of cytochrome P450 (CYP) 3A4 (CTX-4430-DI-001, NCT02233244), a Phase 1 mass-balance and metabolism study (CTX-4430-ADME-001), and 2 Phase 2 studies: a multiple-dose study for 12 weeks in subjects age 16-44 with moderate to severe facial acne vulgaris (CTX-4430-AV-201, NCT02385760), and a multiple-dose study for 48 weeks in CF patients age 18-30 (CTX-4430-CF-201, NCT02443688).

In humans, orally administered Acebilustat is rapidly absorbed, with Tₘₐₓ (time at which maximum plasma concentration was observed) of approximately 1.5 hours when drug was taken while fasting and 5 to 6 hours when drug was taken with a high-fat meal. Observed T_{1/2} (elimination half-life) differed between healthy volunteers (15 to 18 hours) and CF patients (8 to 9 hours) for reasons that are not yet known. Despite the apparent difference in Tin, exposures did not vary appreciably between healthy volunteers and CF patients, as assessed by maximum observed plasma concentration (Cₘₐₓ) and area under the time-concentration curve (AUC). Acebilustat exposure increases to steady state over the first 7 days of treatment. In the clinical drug-drug interaction study, steady state exposures of Acebilustat did not induce CYP3A4/5, suggesting that Acebilustat does not alter the pharmacokinetics of other drugs subject to metabolism by CYP3A4/5. Population PK analysis conducted as a sub-study of the Phase 2 CF trial demonstrates a broad range of Acebilustat clearance rates that are dependent on both body size and concomitant medications in CF patients; wherein larger body size and concomitant use of cystic fibrosis transmembrane conductance regulator (CFTR) modulator therapies (ivacaftor or ivacaftor+lumacaftor) both increase clearance, thereby reducing plasma concentrations, of Acebilustat.

Once-daily oral doses up to 200 mg/day for 14 days in healthy volunteers was well tolerated. Acebilustat was generally well tolerated in CF patients at doses of 50 mg and 100 mg per day for 48 weeks in CTX-4430-CF-201. Adverse events (AEs) in these studies were generally mild to moderate, Headache was the most common adverse event seen in the clinical studies with 8% considered to be related to Acebilustat. In addition, infective pulmonary exacerbation was the most frequently observed TEAE (with the incidence being higher on placebo than on Acebilustat) in the CF-201 clinical study. Infective pulmonary exacerbation would be an expected adverse reaction given the context of the CF population. Acebilustat treatment did not alter circulating neutrophil counts or sputum microbiology.

In the 48-week Phase 2 CF study, results of the primary efficacy endpoint analysis of change from baseline in percent predicted FEV₁ showed no statistically significant difference between Acebilustat and placebo. The secondary efficacy analyses showed a concordant numerical benefit in both reduced rate of pulmonary exacerbations and increased time to first pulmonary exacerbation from treatment with Acebilustat. Additionally, clinically meaningful numerical benefits in reduced rate of pulmonary exacerbations and increased time to first pulmonary exacerbation were observed in the pre-specified subgroups of subjects with milder disease (baseline ppFEV1 >75) and in subjects on concomitant CFTR modulator therapy. Once-daily oral doses of 50 mg and above in humans demonstrated significant engagement of the pharmacologic mechanism in blood over the entire 24-hour dosing interval and throughout the dosing period in phase 1 studies. However, in the Phase 2 CF study, a once daily dose of 100 mg provided more consistent engagement of the pharmacologic mechanism over the 24-hour dosing interval compared to the 50 mg dose. This was particularly notable for CF patients of larger body size and those taking concomitant CFTR modulator therapy. Thus, 100 mg once daily is the current recommended dose for clinical proof of concept studies.

### 2. Study Design

We propose to enroll 70 adult patients with unilateral breast cancer lymphedema. The potential subjects will be recruited from the Stanford Center for Lymphatic and Venous Disorders, a high-volume, focused specialty clinic that has previously been utilized for successful enrollment for prior, similar clinical studies. The design will utilize a 12-week placebo run-in, with assessment of study endpoints before and after placebo exposure. The participants will be informed that they will receive a placebo for Acebilustat for 12 of the 36 weeks of active treatment, but they will be blinded to the exact timeframe of the 12-week placebo exposure (all will receive placebo during Weeks 0-12). This design will permit statistical analysis of the subjective response to placebo and facilitate statistical comparison of the subjective responses to placebo and active drug, respectively. All participants will receive open-label use of Acebilustat. Pre- and post-treatment evaluations will be performed in the Stanford CTRU. The primary endpoint of the study will be change in ultrasonographically measured dermal thickness. The secondary endpoint will be change in caliper skinfold thickness of the affected limb. Exploratory endpoints include assessments of limb volume, and serial assessment of validated patient-reported outcomes, including visual analog scale quantitation of perceived impact of treatment on symptoms and function. Paired comparisons of the placebo run-in and drug responses will be performed. Hepatic function monitoring will be conducted at each patient encounter.

### Correlative Studies Background

We plan correlative studies that will address molecular characterization of the treatment response to LTB4 antagonism in lymphedema. Potential interpretation of these molecular data and their relationship to the treatment response may require correlation with the concomitant changes in prevailing plasma LTB4 concentrations. Accordingly, pre- and post-treatment LTB4 plasma concentrations will be assayed in parallel through ELISA.

With the desire to explicate the molecular pathogenesis of lymphedema, we have previously investigated a murine model of acquired, postsurgical lymphedema¹⁶. In this prior study, the presence of intense histopathological inflammatory changes in the dermis correlated with impaired mobilization of immunocompetent cells from the lymphedematous regions. Large-scale, transcriptional profiling of the lymphedematous tissues disclosed a distinct, predominant inflammatory molecular expression profile that led us to postulate a role for leukotrienes, both in the pathogenesis of the disease and as a potential therapeutic target¹⁶. With these results, and the unresolved need for drug therapy, we decided to investigate the therapeutic impact of ketoprofen¹⁷, an NSAID agent with a recognized dual anti-inflammatory mechanism of action that includes inhibition of the 5-lipoxygenase (5-LO) pathway¹⁸. When we systemically administered ketoprofen to mice with experimental, acquired lymphedema, there was documented reversal of disease burden, including remarkable normalization of the pretreatment lymphedematous histopathology¹⁷. Our subsequent preclinical investigations, both in vitro and in vivo¹⁵ strongly suggest that the therapeutic benefit of ketoprofen in experimental lymphedema is specifically attributable to its inhibition of the 5-LO pathway.

Preclinical investigation of the effects of bestatin, an LTA4H inhibitor, disclosed that, like ketoprofen, LTB4 antagonism reversed edema, improved lymphatic function, and restored lymphatic architecture in the murine model of lymphedema. While low LTB4 concentrations promoted human lymphatic endothelial cell sprouting and growth, higher concentrations (observed in both untreated murine and human lymphedema) inhibited lymphangiogenesis and induced apoptosis. During lymphedema progression, lymphatic fluid LTB4 concentrations rose from initial pro-lymphangiogenic concentrations into an anti-lymphangiogenic range. High concentrations of LTB4 inhibited vascular endothelial growth factor receptor 3 and Notch pathways in cultured human lymphatic endothelial cells. Lymphatic-specific Notch1(−/−) mice were refractory to the beneficial effects of LTB4 antagonism, suggesting that LTB4 suppression of Notch signaling is an important mechanism in disease maintenance. In summary, we found that LTB4 was harmful to lymphatic repair at the concentrations observed in established disease. These findings support the concept that LTB4 is a promising drug target for the treatment of lymphedema.

Our subsequent clinical translational investigation has included a characterization of the inflammatory molecular signature of human lymphedema. Prospective transcriptomic profiling of both murine¹⁶ and human²¹ lymphedema has identified a remarkably small number of specific pathways with altered cutaneous expression in lymphedema. Among the identified central pathways in human lymphedema, the inflammatory substrate appears to be directly linked to the pathogenesis of tissue pathology. In our pilot, placebo-controlled prospective trial of oral ketoprofen therapy for human lymphedema¹⁹, the salutary effects of treatment on both skin thickness and dermal histopathology were accompanied by a distinct alteration in the circulating cytokines and chemokines, as detected by Luminex multiplex assay. We have observed similar patterns of cytokine response in a small, placebo-controlled trial of ubenimex, an LTA4H inhibitor (unpublished observations).

### 3. Participant Selection and Enrollment Procedures

Inclusion Criteria: Unilateral Stage II chronic lymphedema of an upper extremity with an affected: unaffected limb volume ratio of ≥1.2 and duration of ≥6 months. If a potential participant has undergone prior microvascular or debulking surgical intervention, at least one year must have elapsed prior to screening. There are no gender or race-ethnic restriction. Participants ages 18-75 are eligible for enrollment. ECOG performance status will be employed. ECOG Stage 0-2 are eligible. The following statement will be included in the Informed Consent Form: “The participant must have the ability to understand and the willingness to sign a written informed consent document.”

Physical therapy interventions for lymphedema must be completed at least 8 weeks prior to screening. No additional maintenance modalities can be added after screening until the study protocol is completed. Any potential participants who plan elective surgical intervention for lymphedema will be excluded.

No other Investigational Agents will be permitted.

Any other medical condition that could lead to acute limb edema, such as (but not limited to) acute venous thrombosis; other medical condition that could result in symptoms overlapping those of lymphedema in the affected limb (e.g., pain, swelling, decreased range of motion); history of clotting disorder (hypercoagulable state); chronic (persistent) infection in the affected limb; any other infection (unrelated to lymphedema) within 1 month prior to screening; currently receiving chemotherapy or radiation therapy; current evidence of active malignancy, or a history of malignancy within the past 2 year (except for non-melanoma skin cancer or cervical cancer in situ treated with curative intent). If the participant has undergone cancer treatment, this must have been completed >2 year prior to enrollment; chronic renal insufficiency (defined as serum creatinine >2.5 mg/dL or an estimated glomerular filtration rate [eGFR]<30 mL/min at screening) or requires dialytic support; hepatic dysfunction, defined as alanine transaminase (ALT) or aspartate transaminase (AST) levels >3×upper limit of the normal range (ULN) and/or bilirubin level >2×ULN at screening; absolute neutrophil count <1500 mm3 at screening; hemoglobin concentration <9 g/dL at screening; pregnancy or nursing; substance abuse (such as alcohol or drug abuse) within 6 months prior to screening; any reason (in addition to those listed above) that, in the opinion of the investigator, precludes full participation in the study.

Requirements regarding history of allergic reactions attributed to compounds of similar chemical or biologic composition to investigational agent or device: A known allergy to any of the chemical components in the investigational agent.

Agent-specific exclusion criteria: Any current or prior therapeutic use of ketoprofen.

Pregnant and nursing participants will be excluded.

All participants must be provided a consent form describing the study with sufficient information for participants to make an informed decision regarding their participation. Participants must sign the IRB approved informed consent prior to participation in any study specific procedure. The participant must receive a copy of the signed and dated consent document. The original signed copy of the consent document must be retained in the medical record or research file.

Participants will not be randomized.

Primary completion will occur 24 months after first participant accrual. Study completion will occur 30 months after first participant accrual.

### 4. Treatment Plan

### V1 Screening/Visit 1

After obtaining written informed consent, the following activities will occur:
Complete physical examination, including measurements of height, weight, BMI, and vital signs (heart rate, blood pressure, respiratory rate, body temperature), and weight.
Assessment of medications
Assessment of symptoms
Blood and urine samples for clinical laboratory tests including CBC, comprehensive metabolic panel, urinalysis, beta-HCG (premenopausal females only)
Plasma samples will be banked for future correlative studies
LYMQOL questionnaire and VAS scores to assess impact on quality-of-life
Limb volume measurements of both upper extremities
Caliper measurements for skin thickness
Dermal ultrasonography
The medication diary will be collected and pill counts will be performed
The participant will be provided with a 3-month supply of study medication. The participant will be instructed on the exact manner in which to take the medication, and this will commence on the morning after Visit 1. A medication diary will be provided to the participant.
Participant will be instructed to save and return all study medication bottles

### V2 Visit 2 (Week 13±1)

Collect demographic information
Review medical history
Complete physical examination, including measurements of height, weight, BMI, and vital signs (heart rate, blood pressure, respiratory rate, body temperature), and weight
Assessment of medications
Assessment of symptoms
VAS scores to assess impact on quality-of-life
Limb volume measurements of both upper extremities
Caliper measurements for skin thickness
Dermal ultrasonography
The medication diary will be collected and pill counts will be performed
The participant will be provided with the next 3-month supply of study medication and a new medication diary
Participant will be instructed to save and return all study medication bottles

### V3 Visit 3 (Week 25±1)

Collect demographic information
Review medical history.
Complete physical examination, including measurements of height, weight, BMI, and vital signs (heart rate, blood pressure, respiratory rate, body temperature), and weight.
Assessment of medications
Assessment of symptoms
The medication diary will be collected and pill counts will be performed
The participant will be provided with the next 3-month supply of study medication and a new medication diary
Participant will be instructed to save and return all study medication bottles

### V4 Visit 4 (Week 37±1)

Collect demographic information
Review medical history
Complete physical examination, including measurements of height, weight, BMI, and vital signs (heart rate, blood pressure, respiratory rate, body temperature), and weight.
Assessment of medications
Assessment of symptoms
Blood and urine samples for clinical laboratory tests including CBC, comprehensive metabolic panel, urinalysis
Plasma samples will be banked for future investigational use
LYMQOL questionnaire and VAS scores to assess impact on quality-of-life
Limb volume measurements of both upper extremities
Caliper measurements for skin thickness
Dermal ultrasonography
The medication diary will be collected and pill counts will be performed

Participants who discontinue from study early will be asked to return for an early termination visit within 1 week of study drug discontinuation. Any participant who terminates study participation because of an AE will be followed until the outcome of AE is adjudicated.

### 4.1 General Concomitant Medication and Supportive Care Guidelines

The participant will be instructed to avoid prescription ketoprofen for the duration of enrollment.
Phlebotomy (SOC and research): Risks of phlebotomy include: pain; bruising; bleeding; inflammation; infection; temporary redness of the skin where the venipuncture is performed; and light headedness. Care will be taken to avoid these difficulties.
Caliper measurement of skin thickness (research): The calipers may cause some discomfort due to the pinching of the skin.
Limb volume quantitation by circumferential tape measurements (research): no attendant risk
Dermal ultrasonography (research): no attendant risks.
   4.2 Criteria for Removal from Study

It will be documented whether or not each subject completed the clinical study. Participants who discontinue from the study early will be asked to return for a final study visit within the 1 week following the decision to withdraw. If a participant withdraws, all efforts will be made to complete and report the observations, particularly the follow up examinations, as thoroughly as possible. A sincere effort will be made to contact the participant either by telephone, letter, or email, to determine the reason why they failed to return for the necessary visits or withdrew from study, and reason(s) will be documented.

Reasons that a subject may discontinue participation in a clinical study may include the following:
Intercurrent illness that prevents continuation in study
Potential health hazard to patients, as indicated by the incidence or severity of AEs
The subject may choose to withdraw from the study at any time for any reason
General or specific changes in the patient's condition that render the subject unacceptable for further treatment in the judgment of the investigator
Severe non-compliance to protocol as judged by the investigator
Death
Closure of study

### 4.3 Alternatives

The study procedures involve minimal alternatives. If the potential participant chooses not to participate, the alternative would continuation of maintenance physical interventions to stabilize edema.

### 5. Investigational Agent/Device/Procedure Information 5.1 Investigational Agent/Device/Procedure

Acebilustat and matching placebo are provided as size 0 white hard gelatin capsules and supplied in white high-density polyethylene (HDPE) bottles in quantities of 30-36 capsules per unit. Acebilustat capsules contain 100 mg active drug and approximately 250 mg inactive dry powder excipient blend. Placebo capsules contain approximately 325 mg inactive dry powder excipient blend. Acebilustat or placebo capsules are administered orally once-daily with water at breakfast time. Investigational product is stable at controlled room temperature (15 to 25° C./59 to 77° F.) for at least 4 years when protected from exposure to ultraviolet (UV) light.

For this study, placebo is administered orally once-daily with breakfast for 12 weeks, followed by Acebilustat treatment which is administered orally once-daily with breakfast for 24 weeks.

The investigational drug product, Acebilustat and its matching placebo will be provided by Celltaxis LLC.

### 6. Dose Modifications

No dose modifications are feasible for this trial. The Principal Investigator will terminate treatment whenever any of the following conditions are met and the emergent adverse reaction is suspected to be potentially related to treatment:
ALT and/or AST elevated to a level of ≥3× Upper Limit of Normal (ULN)
ALT and/or AST elevated to a level of ≥2.5× ULN and T Bili ≥1.5× ULN
Upon evidence of any Suspected Unexpected Serious Adverse Reaction

### 7. Adverse Events and Reporting Procedure

### 7.1 Potential Adverse Events

A total of 327 subjects across six clinical studies have received at least one dose of acebilustat, including 145 subjects with cystic fibrosis. Acebilustat doses ranged from 5 to 200 mg; 112 subjects with cystic fibrosis. received Acebilustat (at doses of 50 and 100 mg) for 48 weeks. Acebilustat was generally safe and well tolerated across the six clinical studies. In healthy volunteer studies, no clinically significant trends were observed. The table below summarizes the most common treatment emergent adverse events in the safety population:

| | Placebo | | Acebilustat, n (%) | | | | Overall | |
|---|---|---|---|---|---|---|---|---|
| MedDRA System Organ Class | (N = 66) | | 50 mg | | 100 mg | | (N = 199) | |
| Preferred Term | n (%) | | (N = 67) | | (N = 66) | | n (%) | |
| No. of subjects with at least one TEAE | 65 | (98.5%) | 67 | (100.0%) | 66 | (100.0%) | 198 | (99.5%) |
| Gastrointestinal disorders | 26 | (39.4%) | 27 | (40.3%) | 20 | (30.3%) | 73 | (36.7%) |
| Abdominal pain | 4 | (6.1%) | 3 | (4.5%) | 4 | (6.1%) | 11 | (5.5%) |
| Abdominal pain upper | 5 | (7.6%) | 7 | (10.4%) | 3 | (4.5%) | 15 | (7.5%) |
| Constipation | 3 | (4.5%) | 4 | (6.0%) | 1 | (1.5%) | 8 | (4.0%) |
| Diarrhoea | 4 | (6.1%) | 9 | (13.4%) | 5 | (7.6%) | 18 | (9.0%) |
| Distal intestinal obstruction | 3 | (4.5%) | 4 | (6.0%) | 2 | (3.0%) | 9 | (4.5%) |
| syndrome | | | | | | | | |
| Nausea | 5 | (7.6%) | 5 | (7.5%) | 8 | (12.1%) | 18 | (9.0%) |
| General disorders and administration | 18 | (27.3%) | 24 | (35.8%) | 24 | (36.4%) | 66 | (33.2%) |
| site conditions | | | | | | | | |
| Chest discomfort | 7 | (10.6%) | 3 | (4.5%) | 8 | (12.1%) | 18 | (9.0%) |
| Fatigue | 7 | (10.6%) | 9 | (13.4%) | 9 | (13.6%) | 25 | (12.6%) |
| Pyrexia | 7 | (10.6%) | 6 | (9.0%) | 8 | (12.1%) | 21 | (10.6%) |
| Infections and infestations | 58 | (87.9%) | 59 | (88.1%) | 60 | (90.9%) | 177 | (88.9%) |
| Gastroenteritis | 2 | (3.0%) | 1 | (1.5%) | 4 | (6.1%) | 7 | (3.5%) |
| Influenza | 4 | (6.1%) | 3 | (4.5%) | 5 | (7.6%) | 12 | (6.0%) |
| Nasopharyngitis | 13 | (19.7%) | 11 | (16.4%) | 14 | (21.2%) | 38 | (19.1%) |
| Rhinitis | 2 | (3.0%) | 4 | (6.0%) | 3 | (4.5%) | 9 | (4.5%) |
| Sinusitis | 5 | (7.6%) | 3 | (4.5%) | 7 | (10.6%) | 15 | (7.5%) |
| Investigations | 23 | (34.8%) | 20 | (29.9%) | 23 | (34.8%) | 66 | (33.2%) |
| Alanine aminotransferase increased | 2 | (3.0%) | 0 | | 4 | (6.1%) | 6 | (3.0%) |
| Forced expiratory volume decreased | 4 | (6.1%) | 4 | (6.0%) | 5 | (7.6%) | 13 | (6.5%) |
| Sputum abnormal | 1 | (1.5%) | 4 | (6.0%) | 0 | | 5 | (2.5%) |
| Musculoskeletal and connective tissue | 9 | (13.6%) | 13 | (19.4%) | 11 | (16.7%) | 33 | (16.6%) |
| disorders | | | | | | | | |
| Arthralgia | 1 | (1.5%) | 3 | (4.5%) | 5 | (7.6%) | 9 | (4.5%) |
| Back pain | 1 | (1.5%) | 4 | (6.0%) | 1 | (1.5%) | 6 | (3.0%) |
| Nervous system disorders | 14 | (21.2%) | 22 | (32.8%) | 15 | (22.7%) | 51 | (25.6%) |
| Headache | 7 | (10.6%) | 16 | (23.9%) | 9 | (13.6%) | 32 | (16.1%) |
| Respiratory, thoracic and mediastinal | 40 | (60.6%) | 47 | (70.1%) | 50 | (75.8%) | 137 | (68.8%) |
| disorders | | | | | | | | |
| Cough | 22 | (33.3%) | 27 | (40.3%) | 29 | (43.9%) | 78 | (39.2%) |
| Dyspnoea | 5 | (7.6%) | 6 | (9.0%) | 5 | (7.6%) | 16 | (8.0%) |
| Epistaxis | 1 | (1.5%) | 4 | (6.0%) | 3 | (4.5%) | 8 | (4.0%) |
| Haemoptysis | 11 | (16.7%) | 17 | (25.4%) | 12 | (18.2%) | 40 | (20.1%) |
| Oropharyngeal pain | 4 | (6.1%) | 7 | (10.4%) | 9 | (13.6%) | 20 | (10.1%) |
| Paranasal sinus hypersecretion | 1 | (1.5%) | 5 | (7.5%) | 0 | | 6 | (3.0%) |
| Rales | 1 | (1.5%) | 1 | (1.5%) | 4 | (6.1%) | 6 | (3.0%) |
| Rhinorrhoea | 2 | (3.0%) | 6 | (9.0%) | 5 | (7.6%) | 13 | (6.5%) |
| Sputum increased | 11 | (16.7%) | 13 | (19.4%) | 7 | (10.6%) | 31 | (15.6%) |
| Skin and subcutaneous tissue disorders | 8 | (12.1%) | 8 | (11.9%) | 16 | (24.2%) | 32 | (16.1%) |
| Rash | 1 | (1.5%) | 3 | (4.5%) | 6 | (9.1%) | 10 | (5.0%) |

No SARs were reported in the Phase 1 studies in healthy volunteers or subjects with CF.

SARs reported in the completed Phase 2 studies and suspected to be potentially related to drug treatment are tabulated below.

### Serious Adverse Reactions by System Organ Class and Preferred Term: Phase 2

### Acebilustat Safety Population

| | # of | | Intensity | | |
|---|---|---|---|---|---|
| System Organ Class | Subjects | Frequen- | | Moder- | Se- |
| Preferred Term^{a} | N = 217 | cy | Mild | ate | vere |
| Gastrointestinal disorders | 1 | 0.5% | 0 | 0 | 1 |
| Distal intestinal obstruction | 1 | 0.5% | 0 | 0 | 1 |
| syndrome | | | | | |
| Hepatobiliary Disorders | 1 | 0.5% | 0 | 0 | 1 |
| Drug Induced Liver Injury | 1 | 0.5% | 0 | 0 | 1 |
| Infections and infestations | 1 | 0.5% | 0 | 0 | 1 |
| Infective pulmonary | 1 | 0.5% | 0 | 0 | 1 |
| exacerbation of cystic | | | | | |
| fibrosis | | | | | |

^{a}All adverse event (AE) terms were coded using Medical Dictionary for Regulatory Activities (MedDRA) Versions 17.1 and 18.0.

### 8. Correlative/Special Studies

### 8.1 Laboratory Correlative Studies

### 8.1.1 Molecular Characterization of the Treatment Response to LTB4 Antagonism in Lymphedema.

Upon completion of all end of study clinical events, we will undertake a molecular analysis of the pre- and post-treatment samples collected during the trial. The intent is to characterize the molecular pathway responses to treatment and to potentially identify pre-treatment variables that can predict a salutary response to treatment. Four sets of analyses will be performed, comprising a multi-omics approach:
1. Plasma concentration of LTB4 will be determined using Leukotriene B4 ELISA kits from Cayman Chemicals (#520111).
2. Specific expression of inflammatory cytokines and chemokines will be assessed using Bio-rad Luminex human 48-plex panel kits (12007283).
3. Extracellular vesicles will be purified from blood plasma by ultracentrifugation. Purity, size, and concentrations of vesicles will be evaluated using Nanosight NS300 (Malvern). Constitutions of these vesicles are determined by mass spectrometry. The exosome analysis for protein biomarkers will be performed as previously described 22.
4. Multiple panel flow cytometry analysis will be performed to assess the cellular components in PBMCs.
5. Single cell RNA sequencing will be employed to characterize how the impact of LTB4 antagonism upon the transcriptomic landscape of various immune populations.
   8.1.1.1 Collection of Specimen(s): Two phlebotomies will be performed: the first at enrollment and the second at end of study. Whole blood will be collected in CPT tubes and mixed by gently inverting the tubes five to ten times.
   8.1.1.2 Handling of Specimens(s): To preserve bio-stability of LTB4 and its metabolites, plasma and buffy coat are immediately separated through centrifugation at room temperature in a horizontal rotor at 1800 g for 30 minutes. Plasma will be pipetted and aliquoted (1 ml each) into clean screw-capped vials. Individual plasma samples will then be snap-frozen and transported to the −80° C. freezer of the research laboratory.

Peripheral blood mononuclear cells from the buffy coat will be transferred into a 50 ml conical tube. Three cell volumes of phosphate buffered saline (PBS) are added into the tube, mixed with cells, and centrifuged at room temperature for 10 minutes at 300 g. The PBS addition, mixing and centrifuge steps are repeated X 3 and a cell count is performed. At the end of the last centrifuge step, the cell pellet is suspended in the freezing media at 5×10⁶ cell/ml. One ml aliquots of cell suspension are placed into pre-labeled cryovials. The vials are stored in cell freezing containers at −80° C.

8.1.1.3 Site(s) Performing Correlative Study: The correlative studies will be performed in the Stanford research laboratory of the investigators. Plasma aliquots will be stored in a −80° C. freezer. PBMCs will be stored in liquid nitrogen.
8.1.1.4 Coding of specimens for privacy protection: Plasma samples for correlation research purposes will be identified with a unique study identifier. The research team members will de-identify participant information. All identifiers (e.g., name, initials medical record numbers, accession numbers) will be removed. Only the research team will have access to the specimens. A study participant number will be assigned a number once the participant has signed the informed consent. Documents that include the name of the participant will be securely maintained by the Investigator and research team. The key to the code will be maintained by the study protocol director and the study coordinator, in a password protected Stanford computer kept in a secure location, not accessible to the public. Research staff are required to complete HIPAA certification. In addition, study coordinators are required to complete additional training in Good Clinical Practice Guidelines.

### 9. Study Calendar/Schedule of Assessments

| | | Wk | Wk | Wk | WK |
|---|---|---|---|---|---|
| | Pre-Study | 0 | 13 ± 1 | 25 ± 1 | 37 ± 1 |
| Investigational | | X | X | X | |
| Agent | | | | | |
| Informed consent | X | | | | |
| Demographics | X | | | | |
| Medical history | X | | | | |
| Concurrent meds | X | X----------------------------------------------------------------------------------------------X | | | |
| Physical exam | X | | X | X | X |
| Vital signs | X | | X | X | X |
| Height | X | | | | |
| Weight | X | | X | X | X |
| Performance status | X | | X | X | X |
| CBC w/diff, plts | X | | | | X |
| Serum chemistry^{b} | | | | | |
| Research blood | X | | | | X |
| collection | | | | | |
| Adverse event | | X--------------------------------------------------------------------------------------------X | | | |
| evaluation | | | | | |
| B-HCG | X^{c} | | | | |
| Dermal | | X | X | | X |
| ultrasonography | | | | | |
| Caliper skinfold | | X | X | | X |
| thickness | | | | | |
| Limb volume | | X | X | | X |
| Quality of life | X | | X | | X |
| assessments | | | | | |

a: Investigational Agent: Dose as assigned; route/schedule.
^{b}Albumin, alkaline phosphatase, total bilirubin, bicarbonate, BUN, calcium, chloride, creatinine, glucose, LDH, phosphorus, potassium, total protein, SGOT[AST], SGPT[ALT], sodium.
^{c}Serum pregnancy test (women of childbearing potential).
d: Off-study evaluation.

### 10. Measurements

Primary Outcome Measure Change ultrasonographic measurement of dermal thickness in the affected upper extremity
Title: Change in ultrasonographic dermal thickness
Time Frame: Following 36 weeks' enrollment in the protocol
Safety Issue: Is this outcome measure assessing a safety issue? No
   Note: Each outcome measure listed within the protocol will necessitate legally required results reporting to clinicaltrials.gov within one year after the completion of the primary outcome measure.

### 10.1 Primary Outcome

The primary outcome measure is change in ultrasonographic dermal thickness.

10.1.1 Relevant Subset: The target population is participants with unilateral upper extremity lymphedema who are enrolled in this study.

10.1.2 Measurement Definition: This is an objective efficacy measure. The measurement of dermal thickness at enrollment will be compared to the measurement at Week 12, and the measurement at Week 12 will be compared to the measurement at study end (Week 36). We will quantitatively evaluate the difference in ultrasonographic dermal thickness measurement from enrollment to Week 12 to that from Week 12 to Week 36. Dispersion (variance) will be assessed as the standard deviation.

10.1.3 Measurement Methods: Dermal thickness will be derived from an ultrasonographic examination of the skin of the forearm using a Terason 3200T device. The region of interest will be identified on first examination at enrollment, and the same region of interest will be re-interrogated at each of the subsequent examinations. For final interpretation, the assessor will be blinded to participant identity and treatment status. Pre and post-treatment measurement changes will be utilized and reported.

10.1.4 Measurement Time Points: Ultrasonographic dermal thickness will be measured at enrollment, at Week 12 and at end of study (Week 36).

10.1.5 Response Review: Results will be evaluated by a designated, independent, blinded reviewer.

### 10.2 Secondary Outcome

Change in measurement of dermal thickness, as measured by caliper skin fold thickness.

10.2.1 Relevant Subset: The target population is participants with unilateral upper extremity lymphedema who are enrolled in this study.

10.2.2 Measurement Definition: This is an objective efficacy measure. The Measurement of skin thickness at enrollment will be compared to the measurement at Week 12 and the measurement at end of study (Week 36) study end. This measurement is defined as the arithmetic mean of values (with standard deviation) obtained at the two qualifying screening visits. We will quantitatively evaluate the difference in skinfold thickness measurement from enrollment to Week 12 to that from Week 12 to Week 36. Dispersion (variance) will be assessed as the standard deviation.

10.2.3 Measurement Methods: Skin thickness measurements (in mm) will be performed with a Lange skinfold caliper (Beta Technology, Santa Cruz, CA). For each participant, at each assessment, three measurements will be obtained: the dorsum of the hand, the midpoint of the volar aspect of the forearm, and the midpoint of the medial aspect of the upper arm. At the initial evaluation, a dermatographic pencil is used to mark the site of each measurement. Once the locations are determined, the location is noted (measured from the wrist). These locations will be re-utilized for serial measurements for each follow-up visit. Calipers are calibrated prior to each use. Assessor will be blinded to treatment status. Pre- and post-treatment measurement changes will be utilized and reported.

10.2.4 Measurement Time Points: Skin thickness measurements will be performed at enrollment, Week 12 and at end of study (Week 36).

10.2.5 Response Review: Results will be evaluated by a designated, independent, blinded reviewer.

10.3 Exploratory Outcomes 10.3.1 Change in lymphedema Quality of Life (LymQOL): Change in lymphedema Quality of Life (LymQOL) aggregate score.

10.3.1.1 Relevant Subset: The target population is participants with unilateral upper extremity lymphedema who are enrolled in this study.

10.3.1.2 Measurement Definition: This is an objective efficacy measure. The LymQoL survey is a validated, self-reported outcome questionnaire 23. Questions cover four domains: symptoms, body image/appearance, function and mood. Answers are scored 1 to 4 (less severe to severe impairment). LymQOL survey will be used to assess change as a result of the treatment intervention. Change=Week 36 aggregate score-enrollment aggregate score). 10.3.1.3 Measurement Methods: The LymQoL will be completed prior to performance of efficacy assessments (volume measurements, skin caliper measurements, dermal ultrasonography).

10.3.1.4 Measurement Time Points: Participants will complete the LymQoL survey at enrollment and end of study (Week 36).

10.3.1.5 Response Review: Results will be evaluated by a designated, independent, blinded reviewer.

### 10.3.2 Change in VAS Assessment of Symptomatology

Change in VAS scores. In prior clinical investigations of LTA4H inhibition, we have observed significant subjective improvement in the participants who received active drug. The unprompted reports of subjective improvement include the following variables:
Lymphedema limb feels lighter
Sensation of improved lymph flow
Softer skin and tissue
Reduced numbers of infections
Reduced requirement for self-care
Maintenance of lymphedema with lesser grades of compression
Edema became more responsive to treatment
Improved fit of clothing
Improved self-confidence, self-esteem
Decreased embarrassment
Life is more enjoyable

Accordingly, we have constructed and validated a set of VAS analog scales for the assessment of the subjective responses to therapeutic intervention, and these will be serially administered to participants in this study.

10.3.2.1 Relevant Subset: The target population is participants with unilateral upper extremity lymphedema who are enrolled in this study.

10.3.2.2 Measurement Definition: This is an objective efficacy measure. The patient will self-report symptomatology related to lymphedema as expressed on a visual analog scale (VAS). The result of each of these self-reported events will be represented as a linear dimension of −10 to +10, in centimeters (cm). A value of −10 represents the most severe expression of the symptom, and +10 represents the absence of the symptom. Changes will be evaluated at Weeks 12 and 36, and calculated as Week 12 score-enrollment score, or Week 36 score-Week 12 score, respectively. In addition, at Weeks 12 and 36, specific VAS scales will be administered to assess the subjective perception of change in the symptomatic domains.

10.3.2.3 Measurement Methods: The VAS scoring will be completed prior to performance of efficacy assessments (volume measurements, skin caliper measurements, dermal ultrasonography).

10.3.2.4 Measurement Time Points: Participants will complete the VAS scores at enrollment Week 12 and end of study (Week 36).

10.3.2.5 Response Review: Results will be evaluated by a designated, independent, blinded reviewer.

10.3.3 Change in lymphedema limb volume: Change in the volume of the lymphedema affected limb.

10.3.3.1 Relevant Subset: The target population is participants with unilateral upper extremity lymphedema who are enrolled in this study.

10.3.3.2 Measurement Definition: Quantitative assessment of limb volume (mL) of the affected limb at Week 12 will be compared to the enrollment value, and limb volume (mL) of the affected limb at end of study (Week 36) will be compared to the value at Week 12. The mean differences at Week 12 and Week 36 will be compared.

10.3.3.3 Measurement Methods: Limb volume quantification of affected and non-affected limbs, will be performed using circumferential measurements of the limb at 4 cm intervals, beginning at the wrist. The limb volume will be determined using a truncated cone approximation {(volume=πh(R²+Rr+r²)/3), where h is length along the limb axis, R the radius of the lower base, and r the radius of the upper aspect of the truncated cone}. Limb circumference, for calculation of limb volumes, will be serially measured at enrollment, Week 12 and end of study (Week 36). For quantitative analysis, the excess limb volume is defined as the difference in measured volume of the affected and unaffected arms, and changes in excess limb volume will be calculated both as an absolute value and as a percent change.

10.3.3.4 Measurement Time Points: Limb circumference, for calculation of limb volumes, will be measured at enrollment, Week 12 and end of study (Week 36).

10.3.3.5 Response Review: Results will be evaluated by a designated, independent, blinded reviewer.

### 11. Regulatory Considerations

### 11.1 Institutional Review of Protocol

The protocol, the proposed informed consent and all forms of participant information related to the study (e.g., advertisements used to recruit participants) will be reviewed and approved by the Stanford IRB and if applicable Stanford Cancer Institute Scientific Review Committee (SRC). Any changes made to the protocol will be submitted as a modification and will be approved by the IRB prior to implementation. The Protocol Director will disseminate the protocol amendment information to all participating investigators.

### 11.2 Data and Safety Monitoring Plan

The DSMB will audit study-related activities to determine whether the study has been conducted in accordance with the protocol, local standard operating procedures, FDA regulations, and Good Clinical Practice (GCP). This may include review of the following types of documents participating in the study: regulatory binders, case report forms, eligibility checklists, and source documents. In addition, the DSMB will regularly review serious adverse events and protocol deviations associated with the research to ensure the protection of human subjects. Results of the DSMB audit will be communicated to the IRB and the appropriate regulatory authorities at the time of continuing review, or in an expedited fashion, as needed.

### 11.3 Data Management Plan

The Protocol Director and his research team will prepare and maintain adequate and accurate participant case histories with observations and data pertinent to the study. Study specific Case Report Forms (CRFs) will document treatment outcomes for data analysis. Case report forms will be developed using the Redcap or Oncore database system and will be maintained by Principal Investigator and his research team. CRFs (paper documents) will be kept in a secure, locked location, not accessible to the public.

### 12. Statistical Considerations

### Statistical Design

This is designed as an open-label study, insofar as all participants will receive the designated 24-weeks of exposure to the active drug. The participants will be aware that they will also receive a matched placebo during 12 of the 36 weeks of total enrollment, but they are blinded to the fact that the placebo exposure will occur uniformly in each participant during Weeks 0-12 of enrollment. This single-blind design permits intrasubject comparison of the placebo response to the response to active drug.

### Primary Outcome

The primary outcome is designated as the change in ultrasonographically detected dermal thickness in the forearm of the lymphedema-affected upper extremity. The measured change from enrollment to Week 12 will be statistically compared to the measured change from Week 12 to Week 36.

### Secondary Outcome

The secondary outcome is designated as the change in caliper skinfold thickness in the lymphedema-affected upper extremity. The measured change from enrollment to Week 12 will be statistically compared to the measured change from Week 12 to Week 36.

### Primary Analysis

Analysis Population: The primary analysis will include the participants who have completed all required study visits. However, for participants who do not complete the study, their data will be censored and scrutinized with the analysis population. Treatment summary and adverse event data will also be provided for each treatment category separately.

Analysis Plan: The final analysis will be undertaken after the final enrolled participant completes all planned study events. The primary analysis will use all enrolled subjects who have completed the protocol to end of study.

In prospective clinical trials such as the one proposed, a major concern is loss to follow up. In such cases, if a participant is lost to follow up, the participant may be included in the analysis if at least one post-baseline value is recorded prior to loss to follow up. An approach to loss of follow up relies on a flexible assumption about the missing data, namely that the missing values are missing at random. However, in the context of the current proposal, our prior experience with clinical antagonism of 5-LO and LTB4, it is apparent that the clinical response is not measurable prior to an aggregate 6-months of continuous therapy. For this reason, no interim data will be generated or available for analysis in the context of loss to follow up.

For this analysis, we will perform two sets of two-sided paired t-test analyses of the mean change in ultrasonographically-measured dermal thickness that is detected from enrollment to Week 12 (single-blind placebo exposure), to the mean change that is detected from Week 12 to end of study (single-blind exposure to Acebilustat).

### Secondary Analysis

Analysis Population: The secondary analysis will include the participants who have completed all required study visits. However, for participants who do not complete the study, their data will be censored and scrutinized with the analysis population. Treatment summary and adverse event data will also be provided for each treatment category separately.

Analysis Plan: The final analysis will be undertaken after the final enrolled participant completes all planned study events. The secondary analysis will use all enrolled subjects who have completed the protocol to end of study.

In prospective clinical trials such as the one proposed, a major concern is loss to follow up. In such cases, if a participant is lost to follow up, the participant may be included in the analysis if at least one post-baseline value is recorded prior to loss to follow up. An approach to loss of follow up relies on a flexible assumption about the missing data, namely that the missing values are missing at random. However, in the context of the current proposal, our prior experience with clinical antagonism of 5-LO and LTB4, it is apparent that the clinical response is not measurable prior to an aggregate 6-months of continuous therapy. For this reason, no interim data will be generated or available for analysis in the context of loss to follow up.

For this analysis, we will perform two sets of two-sided paired t-test analyses of the mean change in caliper skinfold thickness that is detected from enrollment to Week 12 (single-blind placebo exposure), to the mean change that is detected from Week 12 to end of study (single-blind exposure to Acebilustat).

### Sample Size

Accrual estimates: The Principal Investigator directs a national referral center for lymphatic disorders and sees approximately 600 new patients with clinically significant lymphedema on a yearly basis. Some of these patients are referred from the surgical and oncology breast clinics at Stanford, but majority comes from local and regional medical centers. With this pool of eligible patients, we expect to be able to accrue the required 70 participants within one calendar year. Once the proposed study has been approved and is open to enrollment, we will facilitate enrollment through an IRB-approved marketing campaign for the trial.
Sample size justification: The null hypothesis for this investigation is that treatment of arm lymphedema with Acebilustat for 24 weeks provides no improvement in skin thickness compared to placebo exposure. The alternative hypothesis is that Acebilustat does provide a statistically significant amelioration compared to placebo. For the investigation, the primary and secondary endpoints represent matched pairs of continuous response variables. Prior data, derived from our published, placebo-controlled pilot investigation of ketoprofen therapy in lymphedema¹⁹ indicate that the differences in matched pairs of such data are normally distributed with standard deviation of 6. If the true difference in the mean response of matched pairs is 2.4, we will need to study 68 pairs of subjects to be able to reject the null hypothesis that this response difference is zero with probability (power) 0.9. The Type I error probability associated with this test of this null hypothesis is 0.05.
Effect size justification: The previously demonstrated effect size of LTB4/5-LO antagonism in chronic lymphedema has been demonstrated to provide meaningful subjective and functional improvement in prior participating treatment responders. The current study is designed to replicate or potentially augment the previously observed effect size.
Criteria for future studies: If the current pilot investigation statistically achieves the primary endpoint, we will proceed with an appropriately powered, fully randomized placebo-controlled trial of Acebilustat in chronic lymphedema. Additional prospective clinical investigation will be planned to investigate the capacity for effective preventive therapy with Acebilustat in cohorts at high risk for lymphedema development, as well as formal investigation of the potential for this therapy to reduce the post-treatment incidence of soft tissue infection.

### REFERENCES

1. Brayton K M, Hirsch A T, P J O B, Cheville A, Karaca-Mandic P and Rockson S G. Lymphedema prevalence and treatment benefits in cancer: impact of a therapeutic intervention on health outcomes and costs. PLOS One. 2014; 9: e114597.
2. Karaca-Mandic P, Hirsch A T, Rockson S G and Ridner S H. The Cutaneous, Net Clinical, and Health Economic Benefits of Advanced Pneumatic Compression Devices in Patients With Lymphedema. JAMA Dermatol. 2015; 151:1187-93.
3. Lerman M, Gaebler J A, Hoy S, Izhakoff J, Gullett L, Niecko T, Karaca-Mandic P, O'Donnell T and Rockson S G. Health and economic benefits of advanced pneumatic compression devices in patients with phlebolymphedema. J Vasc Surg. 2018.
4. Lopez M, Roberson M L, Strassle P D and Ogunleye A. Epidemiology of Lymphedema-related admissions in the United States: 2012-2017. Surg Oncol. 2020; 35:249-253.
5. Burian E A, Karlsmark T, Franks P J, Keeley V, Quere I and Moffatt C J. Cellulitis in chronic oedema of the lower leg: an international cross-sectional study. Br J Dermatol. 2021.
6. Rockson S G and Rivera K K. Estimating the population burden of lymphedema. Ann N Y Acad Sci. 2008; 1131:147-54.
7. Rockson S G. Lymphedema after Breast Cancer Treatment. N Engl J Med. 2018; 379:1937-1944.
8. Rockson S G. Lymphatic Medicine: Paradoxically and Unnecessarily Ignored. Lymphat Res Biol. 2017; 15:315-316.
9. Szuba A and Rockson S. Lymphedema: anatomy, physiology and pathogenesis. Vascular Medicine. 1997; 2:321-6.
10. Rockson S G. Update on the biology and treatment of lymphedema. Curr Treat Options Cardiovasc Med. 2012; 14:184-92.
11. Mortimer P S and Rockson S G. New developments in clinical aspects of lymphatic disease. J Clin Invest. 2014; 124:915-21.
12. Rockson S G, Keeley V, Kilbreath S, Szuba A and Towers A. Cancer-associated secondary lymphoedema. Nat Rev Dis Primers. 2019; 5:22.
13. Zampell J C, Avraham T, Yoder N, Fort N, Yan A, Weitman E S and Mehrara B J. Lymphatic function is regulated by a coordinated expression of lymphangiogenic and anti-lymphangiogenic cytokines. Am J Physiol Cell Physiol. 2012; 302: C392-404.
14. Jiang X, Nicolls M R, Tian W and Rockson S G. Lymphatic Dysfunction, Leukotrienes, and Lymphedema. Annu Rev Physiol. 2018; 80:49-70.
15. Tian W, Rockson S G, Jiang X, Kim J, Begaye A, Shuffle E M, Tu A B, Cribb M, Nepiyushchikh Z, Feroze A H, Zamanian R T, Dhillon G S, Voelkel N F, Peters-Golden M, Kitajewski J, Dixon J B and Nicolls M R. Leukotriene B4 antagonism ameliorates experimental lymphedema. Sci Transl Med. 2017; 9.
16. Tabibiazar R, Cheung L, Han J, Swanson J, Beilhack A, An A, Dadras S S, Rockson N, Joshi S, Wagner R and Rockson S G. Inflammatory manifestations of experimental lymphatic insufficiency. PLOS Med. 2006; 3: c254.
17. Nakamura K, Radhakrishnan K, Wong Y M and Rockson S G. Anti-inflammatory pharmacotherapy with ketoprofen ameliorates experimental lymphatic vascular insufficiency in mice. PLOS One. 2009; 4: c8380.
18. Ambrus J L, Jr., Hanciwich S, Chesky L, McFarland P and Engler R J. Improved in vitro antigen-specific antibody synthesis in two patients with common variable immunodeficiency taking an oral cyclooxygenase and lipoxygenase inhibitor (ketoprofen). J Allergy Clin Immunol. 1991; 88:775-83.
19. Rockson S G, Tian W, Jiang X, Kuznetsova T, Haddad F, Zampell J, Mehrara B, Sampson J P, Roche L, Kim J and Nicolls M R. Pilot studies demonstrate the potential benefits of antiinflammatory therapy in human lymphedema. JCI Insight. 2018; 3.
20. Rao N L, Riley J P, Banic H, Xue X, Sun B, Crawford S, Lundeen K A, Yu F, Karlsson L, Fourie A M and Dunford P J. Leukotriene A (4) hydrolase inhibition attenuates allergic airway inflammation and hyperresponsiveness. Am J Respir Crit Care Med. 2010; 181:899-907.
21. Lin S, Kim J, Lee M J, Roche L, Yang N L, Tsao P S and Rockson S G. Prospective transcriptomic pathway analysis of human lymphatic vascular insufficiency: identification and validation of a circulating biomarker panel. PLOS One. 2012; 7: e52021.
22. Ma W, Gil H J, Escobedo N, Benito-Martin A, Ximenez-Embun P, Munoz J, Peinado H, Rockson S G and Oliver G. Platelet factor 4 is a biomarker for lymphatic-promoted disorders. JCI Insight. 2020.
23. Keeley V, Crooks S, Locke J, Veigas D, Riches K and Hilliam R. A quality of life measure for limb lymphoedema. J Lymphoedema. 2010; 5:26-37.

While this invention has been particularly shown and described with references to preferred embodiments thereof, it will be understood by those skilled in the art that various changes in form and details may be made therein without departing from the scope of the invention encompassed by the appended claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference. The relevant teachings of all patents, published applications and references cited herein are incorporated by reference in their entirety.

1. A method treating upper limb extremity lymphedema in a patient in need thereof comprising administering to said patient an effective amount of acebilustat, wherein the treatment results in a reduction in dermal thickening.

2. The method of claim 1, wherein the reduction in dermal thickening is measured by dermal ultrasound.

3. The method of claim 1, wherein the reduction in dermal thickening is measured by skin caliper.

4. The method of claim 1, wherein the patient is suffering secondary or acquired upper limb extremity lymphedema.

5. The method of claim 4, wherein the patient has previously undergone surgery and/or radiation therapy for cancer.

6. The method of claim 5, wherein the cancer is a solid tumor.

7. The method of claim 6, wherein the patient is suffering breast cancer treatment associated upper limb lymphedema.

8. The method of claim 1, wherein the effective amount of acebilustat is less than that which provides maximum inhibition of LTA4H.

9. The method of claim 1, wherein the acebilustat is administered orally.

10. The method of claim 9, wherein the daily dose of acebilustat is 200 mg/day or less.

11. The method of claim 10, wherein the daily dose of acebilustat is 100 mg/day or less.

12. The method of claim 11, wherein the daily dose of acebilustat is 75 mg/day or less.

13. The method of claim 12, wherein the daily dose of acebilustat is 50 mg/day or less.

14. The method of claim 1, wherein the acebilustat is administered twice a day.

15. The method of claim 1, wherein the acebilustat is administered once a day.

16. The method of claim 1, wherein the acebilustat is administered topically.

17. The method of claim 1, wherein the acebilustat is administered by pulsatile dosing or wherein the acebilustat is in a pulsatile release pharmaceutical composition.

18. The method of claim 1, wherein the reduction in dermal thickness occurs within 24 weeks after first administration of acebilustat.

19. The method of claim 1, wherein the reduction in dermal thickening is at least about 10% reduction as compared to baseline.

20. The method of claim 19, wherein the reduction in dermal thickening is at least about 20% reduction as compared to baseline.

21. The method of claim 1, further comprising administering a second active agent, wherein the second active agent is selected from the group consisting of a COX-1 inhibitor, a COX-2 inhibitor, a coumarin, an anti-histamine, montelukast and related leukotriene modifiers, an anti-fibrotic compound, a diuretics, a statin an mTOR inhibitor, and pirfenidone.

22. The method of claim 1, further comprising administering a second active agent, wherein the second active agent is selected from the group consisting of a pro-lymphangiogenic drug, an anti-TGF-b1 antibody, anti-IFN-γ antibody, anti-IL-4 antibody, and anti-IL-13 antibody.

23. A method treating upper limb extremity lymphedema in a patient in need thereof comprising administering to said patient an effective amount of a selective LTA4H inhibitor, wherein the treatment results in a reduction in dermal thickening, wherein the selective LTA4H inhibitor is selective for epoxide hydrolase versus aminopeptidase.

24-31. (canceled)

32. A method of reducing dermal thickening in a patient in need thereof, the method comprising administering to said patient an effective amount of acebilustat or other selective LTA4H inhibitor, wherein the treatment results in a reduction in dermal thickening.

33. The method of claim 32, wherein the patient is in need of improved skin softness or suffering from scleroderma.

34. (canceled)

35. The method of claim 32, wherein the reduction in dermal thickening is measured by dermal ultrasound or skin caliper.

36. (canceled)

37. The method of claim 32, wherein the effective amount of acebilustat is less than that which provides maximum inhibition of LTA4H.

38. The method of claim 32, wherein the acebilustat is administered orally or topically.

39-55. (canceled)
